(19) United States
(12) Patent Application Publication
(10) Pub. No.: US 2011/0197292 A1
(43) Pub. Date: Aug. 11, 2011

(54) Delivery method

(75) Inventor: Bruce A. Sullenger, Durham, NC (US)

(73) Assignee: DUKEUNIVERSITY, Durham, NC (US)

(21) Appl. No.: 12/926,824

(22) Filed: Dec. 10, 2010

Related U.S. Application Data
(63) Continuation of application No. 12/610,330, filed on Nov. 1, 2009.
Application No. 12/610,330 is a continuation-in-part of application No. 12/227,871, filed on Oct. 7, 2009.
(60) Provisional application No. 60/809,842, filed on Jun. 1, 2006.

(51) Int. Cl. A01K 67/00; A61K 31/7088; A61K 31/713; C07H 21/02; C07K 2/00; C07K 16/18; C07K 14/00; C07H 21/04; A61P 35/00; A61P 37/08; A61P 37/00; A61P 29/00; A61P 31/00; C12N 5/10; C07K 14/54; C07K 14/52; C07K 14/555

(52) U.S. Cl. 800/13; 514/44 A; 536/24.5; 530/322; 530/391.7; 530/395; 536/23.1; 514/44 R; 435/375; 435/325; 530/351

(57) ABSTRACT

The present invention relates, in general, to RNA silencing and, in particular, to a method of effecting targeted delivery of an RNA silencing moiety using a targeting moiety that binds to a cell surface receptor and mediates internalization of the RNA silencing moiety to be accessible to Dicer. Also provided is a chimeric nucleic acid molecule comprised of a targeting moiety and an RNA silencing moiety, wherein the targeting moiety is an aptamer and the RNA silencing moiety comprises a Dicer substrate.

This application is a continuation-in-part of U.S. application Ser. No. 12/227,871, filed 1 Dec. 2008, which is the US national phase of International Application No. PCT/US2007/012927, filed 1 Jun. 2007, which designated the US and claims priority to U.S. Provisional Application No. 60/809,842, filed 1 Jun. 2006, the entire contents of which applications are incorporated herein by reference.

This invention was made with Government support under Grant No. R01 HL079051 awarded by the National Institutes of Health. The Government has certain rights in the invention.

### TECHNICAL FIELD

The present invention relates, in general, to interfering RNA (RNAi) (e.g., siRNA, miRNA, and other RNA molecules having a function of silencing or repressing gene expression) and, in particular, to a method of effecting targeted delivery of RNAi's and to compounds suitable for use in such a method.

### BACKGROUND

In a general and brief description of RNA interference (RNAi), endogenously formed (e.g., naturally produced intracellularly) or exogenously introduced (e.g., transfected, or introduced by a microorganism, into a cell) double-stranded RNA (“dsRNA”) is processed by Dicer (an RNAse III family enzyme) into discrete, small RNA duplexes of between 21-25 basepairs. Typically, such duplexes will be referred to as small interfering RNA (siRNA) if the strands of the duplexes are perfectly complementary, or as microRNAs (miRNAs) if the strands of the duplexes are imperfectly base paired (e.g., such as arising from pre-miRNA hairpins). The two strands of such a discrete, small RNA duplex are typically referred to as a “guide” strand, and an “anti-guide” (also referred to as “passenger”) strand. The discrete, small RNA duplexes processed from Dicer are further processed into single stranded RNA where the passenger strand is cleaved, and only the guide strand is loaded into a complex in forming a mature RNA-induced silencing complex known as “RISC”. Mature RISC, assembled with the guide strand of siRNA, is sometimes referred to as “siRISC”, whereas mature RISC assembled with the guide strand of miRNA may be referred to as “mRISC”. Although the mechanisms of RNAi are still being detailed, it appears that when part of a mature RISC, a guide strand (e.g., siRNA or miRNA) that has perfect complementarity with an mRNA can result in mRNA cleavage, whereas a guide strand has less than perfect complementarity (i.e., resulting in base pairing to a portion or portions, but not all, of the mRNA sequence (“partial base pairing”)) may result in translational repression. In either case, the results achieved are termed “RNAi”, or alternatively, “RNA silencing”. In nomenclature commonly used by those skilled in the art, RNA in the form of a dsRNA (guide strand based paired with the passenger strand) or in the form of a single strand (guide strand in RISC), are both typically referred to as RNA silencing moieties. However, in describing the invention herein and in reference to delivery to Dicer, the term “RNA-silencing moiety” is used to mean RNA in the form of a dsRNA (guide strand based paired with the passenger strand).

A critical technical hurdle for RNAi-based clinical applications is the delivery of RNA silencing moieties across the plasma membrane of cells in vivo. A number of solutions for this problem have been described including cationic lipids, viral vectors, high-pressure injection, and modifications of the RNA silencing moieties (e.g. chemical, lipid, steroid, protein). However, most of the approaches described to date have the disadvantage of delivering siRNAs to cells non-specifically, without regard to the cell type (the “major bottleneck in the development of siRNA therapies”, as stated by Aagaard and Rossi, 2007, Adv. Drug Deliv. News 59:75-86). For in vivo use, it is important to target therapeutic RNA silencing moieties to particular cell types (e.g., cancer cells), thereby limiting side-effects that result from non-specific delivery as well as reducing the quantity of RNA silencing moieties necessary for treatment.

The present invention relates to a much simpler approach for specific delivery of RNA silencing moieties and one that, at least in one embodiment, only uses properties of RNA. With SELEX (systematic evolution of ligands by exponential enrichment), it has been demonstrated that structural RNAs capable of binding a variety of proteins with high affinity and specificity can be identified. Thus, using SELEX, an RNA OR DNA molecule may be generated that has the ability to bind a protein on a specific cell type. The delivery method of the instant invention exploits the structural potential of nucleic acids (e.g., RNA) to target a nucleic acid molecule to a particular cell-surface receptor and thus to a specific cell type, combined with the novel discovery of the invention that aptamers by themselves (e.g., without the need for nanoparticles or cationic polymers) can mediate cellular internalization to reach the cytoplasm, and deliver one or more RNA silencing moieties comprising dsRNA to Dicer for processing. The invention thus provides a method to specifically deliver dsRNA into cells by using a targeting moiety binds to a receptor on the cells such binding allows for cellular internalization to deliver dsRNA to the cytoplasm for processing by Dicer. Preferably, the dsRNA comprises a guide strand and a passenger strand. Processing of the delivered dsRNA may then result in RNAi.

### SUMMARY OF THE INVENTION

The present invention relates generally to interfering RNA (RNAi) and to a method of delivering an RNA-silencing moiety comprising dsRNA to Dicer for processing. More specifically, the invention relates to a method of effecting targeted delivery an RNA-silencing moiety comprising dsRNA (e.g., siRNA, miRNA, shRNA, or other RNA-silencing moiety known in the art) that involves the use of a nucleic acid molecule that comprises dsRNA comprising a guide strand to be delivered to Dicer, and a targeting moiety for binding a receptor on a cell with resultant cellular internalization to deliver the dsRNA to the cell cytoplasm to be accessible by Dicer, wherein the targeting moiety is an aptamer. Also provided is a method for making a Dicer substrate, comprising: (a) synthesizing a nucleic acid molecule comprising (i) a targeting moiety comprising an aptamer, and (ii) a first single stranded RNA (e.g., an RNA molecule comprising either a guide strand or a passenger strand) for forming a dsRNA; wherein the targeting moiety and the first single stranded RNA are a contiguous nucleic acid molecule and wherein the targeting moiety is capable of binding to a receptor on the surface of a cell and subsequently being internalized into the cell and locating into the cell cytoplasm; (b) hybridizing a second single stranded RNA, having full complementarity or partial complementarity to the first single stranded RNA, to the first single stranded RNA of the synthesized RNA molecule in forming a dsRNA which can act as a Dicer substrate. The first single stranded RNA and second single stranded RNA may be part of the same RNA strand (e.g., synthesized is a contiguous RNA strand containing a nucleotide sequence that is composed of both the first single stranded RNA and the second single stranded RNA), wherein the RNA strand folds so that the first single stranded RNA and second single stranded RNA undergo base pairing to form a dsRNA. Alternatively, the first single stranded RNA and second single stranded RNA are each produced as separate and discrete RNA strands (e.g., synthesized are two RNA strands, one strand being the first single stranded RNA, and the other strand being the second single stranded RNA) but then the two strands are contacted with each other under conditions effective (as known in the art) to form base pairs (and a duplex) in producing a dsRNA. Also provided is a Dicer substrate produced by this method. The terms “first” and “second”, as used herein for purposes of the specification and claims, to distinguish between two different molecules, or between two different positions on a molecule, as will be more clear from the description.

Also provided is a method of introducing an RNA-silencing moiety comprised of dsRNA into cells comprising contacting the cells with a nucleic acid molecule, wherein the nucleic acid molecule comprises a targeting moiety and a dsRNA molecule, wherein the targeting moiety and at least one strand of the dsRNA molecule are a contiguous nucleic acid molecule, wherein the targeting molecule is an aptamer that recognizes and binds a cell surface receptor on the cells and which becomes internalized into the cells in a process subsequent to binding, and wherein one or more guide strands of the dsRNA becomes bound to Dicer subsequent to introduction of the nucleic acid molecule into the cells.

The methods, and compositions useful for the methods, of the present invention can be practiced in vivo, ex vivo, or in vitro. Objects and advantages of the present invention will be clear from the description that follows.

### BRIEF DESCRIPTION OF THE DRAWINGS

FIG. 1 is a series of diagrams from flow cytometric analyses showing the cell-type specific binding of fluorescently-labeled (FITC) aptamer (A10) and A10 aptamer-siRNA chimeras (A10-CON, A10-Plk1). No binding (dark-shaded peaks) of fluorescently-labeled A10-siRNA chimeras is detected on PSMA-negative cells, PC-3; whereas fluorescently-labeled A10-siRNA chimeras (peaks with no shading) are shown to bind LNCaP cells which express PSMA.

FIG. 2A is a series of diagrams from flow cytometric analyses showing that neither Plk1gene knockdown nor detection of binding/uptake of A10-Plk1 chimera is observed with PC-3 cells contacted with A10-Plk1 chimera.

FIG. 2B is a series of diagrams from flow cytometric analyses showing that decreased (“silenced”) is Plk1 gene expression in LNCaP cells contacted with A10-Plk1chimera as compared to detection of Plk1 gene expression in LNCaP cells contacted with A10-CON chimera, and that binding/uptake of A10-Plk1 chimera is observed with LNCaP cells contacted with A10-Plk1 chimera.

FIG. 3A is a series of diagrams generated by flow cytometric analyses, or immunoblotting analyses (bottom panel), showing detection of PLK1 (protein) levels following transfection of either PC-3 cells or LNCaP cells with siRNAs (CON or Plk1).

FIG. 3B is a series of diagrams generated by flow cytometric analyses, or immunoblotting analyses (bottom panel), showing detection of Bcl2 gene expression levels following transfection of either PC-3 cells or LNCaP cells with siRNAs (CON or Bcl2).

FIG. 3C is a series of diagrams from flow cytometric analyses showing that neither Bcl2 gene knockdown nor detection of binding/uptake of A10-Bcl2 chimera is observed with PC-3 cells contacted with A10-Bcl2 chimera.

FIG. 3D is a series of diagrams from flow cytometric analyses showing that Bcl2 gene expression detection is decreased (“silenced”) in LNCaP cells contacted with A10-Bcl2 chimera as compared to Bcl2 gene expression detection in LNCaP cells contacted with A10-CON chimera, and that binding/uptake of A10-Bcl2 chimera is observed with LNCaP cells contacted with A10-Bcl2 chimera.

FIG. 4 is a diagram showing incorporation of 3H-Thymidine in evaluating cell proliferation, and the effects of transfection of siRNAs (CON, PlK1), or treatment (without transfection) by aptamer A10 or apatmer-siRNA chimeras (A10-CON, A10-PlK1) on cell proliferation in prostate cancer cell lines PC-3 (PSMA−) and LNCaP (PSMA+).

FIG. 5A is a series of diagrams from flow cytometric analysis showing absence or presence of induction of apoptosis (as measured by caspase 3) following treatment of either PC-3 cells or LNCaP cells with cisplatin, Plk1 siRNA, CON siRNA, aptamer A10, and A10 aptamer-siRNA chimeras (A10-CON, A10-Plk1). No detection of caspase 3 is represented by the dark-shaded peaks; whereas induction of caspase 3 is noted with outlined peaks with no shading.

FIG. 5B is a series of diagrams from flow cytometric analysis showing absence or presence of induction of apoptosis (as measured by caspase 3) following treatment of either PC-3 cells or LNCaP cells with cisplatin, Bcl2 siRNA, CON siRNA, aptamer A10, and A10 aptamer-siRNA chimeras (A10-CON, A10-Bcl2). No detection of caspase 3 is represented by the dark-shaded peaks; whereas induction of caspase 3 is noted with outlined peaks with no shading.

FIG. 6 is a series of diagrams from flow cytometric analyses showing that aptamer-siRNA chimera-mediated gene silencing occurs via the RNAi pathway (i.e., utilizing Dicer). LNCaP cells transfected with either siRNAs, A10 aptamer, or A10 aptamer-siRNA chimeras (A10-CON and A10-Plk1) in the presence or absence of an siRNA against Dicer to see the effect of the presence or absence of Dicer on gene silencing of Plk1.

FIG. 7 is a representation of a ethidium bromide-stained polyacrylamide gel showing the effect of the presence or absence of the Dicer enzyme on aptamer-siRNA chimeras (A10-Plk1 and A10-CON), aptamer A10, and single-stranded chimeras (ssA10-Plk1 and ssA10-CON, without antisense siRNA).

FIG. 8 is a representation of an autoradiogram showing the effect of the presence or absence of the Dicer enzyme on aptamer-siRNA chimeras (A10-Plk1 and A10-CON) annealed to the complementary antisense siRNA strand labeled with ³²P, with any resultant cleavage products subsequently resolved on a non-denaturing polyacrylamide gel.

FIG. 9 is a representation of an immunoblot of a PAGE gel for detecting PLK1 (top panel) in LNCaP cells after treatment with transfection reagent alone (lane 1), Plk1 siRNA with transfection reagent (lane 2), or various concentrations of A9-Plk1-chimera (in the absence of transfection reagent; lanes 3-5). Afterwards, the blot was stripped and reanalyzed with antibody specific for beta-tubulin to envisage equal loading of the gel (bottom panel).

FIG. 10 is a graph showing binding of 9.38-Plk1 chimera with c-Kit, as determined by a filter binding assay.

FIG. 11 is a diagram from flow cytometric analysis, with the x-axis showing the intensity of the fluorescence and the y-axis showing the amount of cells at that intensity; the shaded peak represents fluorescence of unstained HeLa cells; the peak outlined with heavy lining represents fluorescence of HeLa cells incubated with Control Aptamer; and the peak outlined with light lining represents fluorescence of HeLa cells incubated with fluorescently labeled AS1411 aptamer.

FIG. 12 is a bar graph representing detection of PLK1 (amount of Plk1 translation), measured from immunoprecipitated radiolabeled protein that was separated on a PAGE gel, in either renal proximal tubule epithelial cells (“RPTEC”) or 786-O cancer cells in cell culture after treatment with an aptamer-siRNA chimera, AS1411-Plk1 chimera.

FIG. 13 is a bar graph representing PLK1 expression after cells were untreated, treated with assay negative controls (“Nucleolin Aptamer-conmiRNA”; “Nucleolin Aptamer-ConsiRNA”) and Aptamer-RNA-silencing moiety chimeras (“Nucleolin Aptamer-Plk1miRNA”; “Nucleolin Aptamer-Plk1siRNA”).

FIG. 14 is a bar graph representing quantification of the levels of active caspases 3 and 7 (“3/7”) (representing measures of apoptosis, and gene knockdown of Plk1) in 786-O primary renal adenocarcinoma cells treated with either AS1411-Plk1 chimera (“Nucleolin Apt-Plk1 siRNA”), AS1411-Plk1misiRNA chimera (“Nucleolin Apt-Plk1 microRNA”) or a respective assay negative control chimera (“Nucleolin Apt-Con siRNA”, or “Nucleolin Apt-Plk1 ConmicroRNA”).

### DETAILED DESCRIPTION OF THE INVENTION

The present invention relates to a method of effecting targeted delivery of RNAs such as RNA silencing moieties (e.g., RNAi's, siRNA, miRNA, and short hairpin RNAs (shRNA), and precursors thereof). This method can be used, for example, to target delivery of siRNA to specific cell types (e.g., cells bearing cell surface receptor, such as a particular protein, carbohydrate or lipid, or combination thereof). In contrast to most delivery methods described to date, the method disclosed herein can be carried out using a compound that comprises only nucleic acid molecule, such as RNA or DNA-RNA. The nucleic acid molecule used is a chimeric molecule (or “chimera”) comprising a nucleic acid targeting moiety (e.g., an aptamer) one or more RNA silencing moieties (e.g., comprising modified or unmodified RNA) in forming a contiguous nucleic acid molecule. In accordance with the invention, the nucleic acid molecule comprising the targeting moiety (e.g., aptamer) can comprise RNA, DNA or any modified nucleic acid-based oligonucleotide (as further described herein).

In one example to illustrate the invention, provided are nucleic acid molecules represented by a targeting moiety-RNA-silencing moiety chimera that: i) specifically binds prostate cancer cells (and vascular endothelium of most solid tumors) expressing the cell-surface receptor prostate specific membrane antigen (“PSMA”), and ii) delivers therapeutic siRNAs (e.g., that target polo like kinase 1 (Plk1) or Bcl2). Plk1 and Bcl2 are known in the art to be two survival genes overexpressed in most human tumors. These chimeric RNA molecules can deliver dsRNA as a substrate for Dicer, thus directing the RNA silencing moiety into the RNAi pathway and silencing their cognate mRNAs. The particular compositions described in this illustrative example may be used in therapeutic applications for treating prostate cancer and other cancers.

Also described is an additional illustrative example, wherein the nucleic acid molecule comprises: another targeting moiety that is also an aptamer that specifically binds to prostate cancer cells via PSMA and is internalized in a process subsequent to binding to PSMA; and an RNA silencing moiety that is ds RNA which comprises siRNA targeting Plk1; wherein the aptamer and the dsRNA (at least one strand thereof) are a contiguous nucleic acid molecule; and wherein the dsRNA (or a portion thereof) becomes a Dicer substrate upon delivery of the nucleic acid molecule into the cells.

In another example to illustrate the invention, provided are nucleic acid molecules comprises a targeting moiety that is an aptamer recognizing a cell surface receptor c-Kit (Stem Cell Factor Receptor); and RNA silencing moiety that is dsRNA comprising siRNA targeting Plk1, wherein the aptamer and the dsRNA (at least one strand thereof) are a contiguous nucleic acid molecule, and wherein the dsRNA (or a portion thereof) becomes a Dicer substrate upon delivery of the nucleic acid molecule into cells expressing a cell surface receptor recognized by the targeting moiety portion of the nucleic acid molecule. Since c-Kit is overexpressed in a variety of cancers (e.g., lung cancer, Ewings Sacroma, chronic myelogenous leukemia, acute myeloid leukemia, and other cancers afflicting humans), the particular compositions described in this illustrative example may be useful in therapeutic applications for treating various types of cancer.

In another example to illustrate the invention, provided are nucleic acid molecules comprises: a targeting moiety that is an aptamer recognizing a cell surface receptor, nucleolin; and an RNA silencing moiety that is ds RNA comprising siRNA targeting Plk1; wherein the aptamer and the dsRNA (at least one strand thereof) are a contiguous nucleic acid molecule; and wherein the dsRNA (or a portion thereof) becomes a Dicer substrate upon delivery of the nucleic acid molecule into cells expressing a cell surface receptor recognized by the targeting moiety portion of the nucleic acid molecule. Since nucleolin is overexpressed, and expressed as a cell-surface receptor, in a variety of cancers (e.g., breast cancer, gliomas, leukemias, angiogenic blood vessels in the tumor environment, prostate cancer, and other cancers afflicting humans), the particular compositions described in this illustrative example may be useful in therapeutic applications for treating various types of cancer.

Thus, the invention described and demonstrated herein is an approach that can be adapted to generate nucleic acid molecules as therapeutics to treat a wide variety of diseases, in addition to cancer. The two requirements for this approach for a given disease are that silencing specific genes in a defined population of cells produces a therapeutic benefit, and that surface receptors are expressed specifically on the cell population or cell type of interest; and that the surface receptor can be utilized (e.g., by its property of recycling, or function) to internalize into cells to deliver RNA ligands intracellularly. As to RNA silencing moieties, and their targets, these are well known in the art (as described herein in more detail). As to delivery, described in detail and demonstrated herein are methods by which one skilled in the art can identify an aptamer that can bind a cell surface receptor and mediate internalization to deliver dsRNA in the cytoplasm to be available as a substrate for Dicer. Many diseases satisfy both of these requirements (examples include, but are not limited to, CD4+ T-cells for HIV inhibition, insulin receptor and diabetes, receptors on liver cells and hepatitis genes, etc). The methods of the present invention can be applied to non-mammalian cells (e.g., animal cells, insect cells, plant cells microbial cells) or mammalian cells, and preferable applications of the methods of the invention are to human cells.

Appropriate targeting and silencing moieties can be designed/selected using methods known in the art based on the nature of the molecule to be targeted and gene(s) to be silenced (see Nimjee et al., Annu. Rev. Med. 56:555-83 (2005) and U.S. Published Application 20060105975). The nucleic acid molecules can be synthesized using any one or more RNA synthesis methods known in the art (e.g. via chemical synthesis, or transcription via RNA polymerases, or recombinant production). Typically, an aptamer is from about 20 nucleotides to about 80 nucleotides, and often is from about 30 nucleotides to 60 nucleotides in length. Short RNA aptamers (e.g., 25-35 bases) that bind various targets with high affinities have been described (Pestourie et al., Biochimie 87(9-10):921-930 (2005), Nimjee et al., supra). Nucleic acid molecules comprising RNA chimeras useful in the present invention that are designed with short aptamers may have a long strand (contiguous strand of aptamer and RNA silencing moiety) of approximately 50 to 60 bases. In producing these nucleic acid molecules, chemically synthesized RNA is amenable to various modifications that can be used to modify its in vivo half-life and bioavailability. (See also, for example, U.S. Application Nos. 20020086356, 20020177570, 20060105975, and 20020055162, and U.S. Pat. Nos. 6,197,944, 6,590,093, 6,399,307, 6,057,134, 5,939,262, and 5,256,555, in addition, see also Manoharan, Biochem. Biophys. Acta 1489:117 (1999); Herdewijn, Antisense Nucleic Acid Drug Development 10:297 (2000); Maier et al., Organic Letters 2:1819 (2000), and references cited therein.) The nucleic acid molecule (the targeting moiety portion, the RNA silencing moiety, or a combination thereof) may comprise modified nucleic acid bases (e.g., modified nucleotides), for example, to improve pharmacokinetics and/or stability (e.g., against nucleases) when administered in vivo. For example, modified purines are know to include, but are not limited to, 2′-O-methyl nucleotides; and modified pyrimidines are known to include, but are not limited to, 2′-deoxy-2′-fluoro nucleotides or 2′-deoxy-2′-fluoroarabino nucleotides. Thus, chemical modifications of nucleotides in producing targeting moieties comprising aptamers may include, without limitation, phosphorothioate internucleotide linkages, 2′-deoxyribonucleotides, 2′-O-methyl ribonucleotides, 2′-deoxy-2′-fluoro ribonucleotides, 4′-thio ribonucleotides, 2′-O-trifluoromethyl nucleotides, 2′-O-ethyl-trifluoromethoxy nucleotides, 2′-O-difluoromethoxy-ethoxy nucleotides, L-nucleotides, and 5-C-methyl nucleotides. Although RNA silencing moieties may comprise long dsRNA (e.g., up to and greater 200 basepairs (bp)), such as useful in non-mammalian systems, in mammalian systems much shorter RNAs of no greater than 30 by are used as RNA silencing moieties so as to (a) reduce possible non-specific effects which may be induced based on the length of the RNA molecule (e.g., unmodified siRNA of 30 or more by have been reported to induce cytokine production, or other cellular process(es) which may not be desired in view of the intended benefit of RNA silencing), and (b) to be substrates for Dicer cleavage at 21-25 bp intervals.

Nucleic acid molecules useful in the methods of the invention can be formulated into pharmaceutical compositions that can include, in addition to the nucleic acid molecule, a pharmaceutically acceptable carrier, diluent or excipient. The precise nature of the composition will depend, at least in part, on the nature of the nucleic acid molecule and the route of administration. Optimum dosing regimens can be readily established by one skilled in the art and can vary with the nucleic acid molecule, the individual being treated, and the effect sought. Generally, the nucleic acid molecule can be administered IV (intravenously), IM (intramuscularly), IP (intraperitoneally), SC (subcutaneously), or topically, as appropriate.

In practice, the targeted delivery method of the instant invention can avoid adverse side-effects associated with delivery of siRNAs to non-targeted cells. For example, siRNAs are known to activate toll-like receptors within plasmacytoid dendritic cells, leading to cytokine secretion, which can result in various adverse symptoms. In the case of delivering siRNAs that trigger apoptosis, another danger that is avoided by use of the present approach is the killing of healthy cells. Treatments involving systemic delivery of nucleic acid molecules useful in the invention can be expected to require substantially less targeted (as compared with non-targeted) RNA silencing moiety due to the reduction in uptake by non-targeted cells. Thus, the method described can also substantially reduce the cost of the. therapy.

As RNA is believed to be less immunogenic than protein, the methods of the present invention can be employed using chimeric RNA molecules (e.g., targeting moiety and RNA silencing moiety) to produce less non-specific activation of the immune system than protein-mediated delivery approaches. This may be an important difference, as a number of proteins currently used for therapeutics are known to occasionally cause dangerous allergic reactions especially following repeated administration.

It has been reported that Dicer-mediated processing of RNAs may result in more efficient incorporation of resulting siRNAs into RISC complexes (Kim et al, Nat. Biotechnol. 23(2):222-6 (2005)). This suggestion is based on the observation that longer double-stranded RNAs (e.g., 25 bp to 29bp), which are processed by Dicer, deplete their cognate mRNAs at lower concentrations than siRNAs (19-21bps), which are not processed by Dicer. Thus, while not wishing to be bound by theory, it is speculated that because the methods of the invention involve delivery to Dicer for processing, the methods may be advantageous (as to delivering RNA silencing moieties which may be more potent in terms of gene-silencing ability) compared to delivery of dsRNA of 19-21 bps that are not processed. Thus, in the methods of the present invention, a targeting moiety recognizes a cell surface receptor on a target cell, internalizes into the cell expressing the receptor, and delivers a RNA silencing moiety to the cytoplasm, wherein the RNA silencing moiety is then accessed and recognized by miRNA- or siRNA-processing machinery (such as Dicer). Processing typically includes cleavage of the RNA silencing moiety to allow it to be loaded into an RNAi or miRNA silencing complex (such as RISC). Thus, at least in a preferred embodiment, the nucleic acid molecules delivered by the method of the invention allow the RNA silencing moiety portion to be recognized and processed by Dicer in a similar way as RNA silencing moieties naturally occurring in cells are recognized and processed by Dicer.

Also, in the methods and compositions of the invention, the RNA silencing moiety portion of the chimeric nucleic acid molecule may comprise more than one guide strand (e.g., each guide strand targeting the same mRNA or a different mRNA) for RNA silencing. For example, a first guide strand and a second guide strand may be on the same RNA strand (synthesized is a contiguous RNA strand containing a nucleotide sequence that is composed of both the first guide strand and the second guide RNA) or be on opposite RNA strands of the RNA silencing moiety, such that upon Dicer processing, produced is more than one guide strand that then may be incorporated into RISC. Nucleotides that are the signal for Dicer processing (as known to those skilled in the art) can be spaced apart between the two or more guide strands to direct Dicer to cleave the dsRNA to produce two or more guide strands for each guide strand to then be incorporated into RISC.

Certain aspects of the invention can be described in greater detail in the non-limiting Examples that follows.

### EXAMPLE 1

As well known in the art, nucleic acid aptamers can be generated by in vitro screening of complex nucleic-acid based combinatorial shape libraries (e.g., >10¹⁴ shapes per library) employing a process termed SELEX (for Systematic Evolution of Ligands by EXponential Enrichment). SELEX is an iterative process in which a library of randomized pool of RNA sequences is incubated with a selected protein-containing target, such as a cell surface receptor of interest which is isolated from cells using methods known in the art. RNA binding to the isolated cell surface receptor is then partitioned from non-binding RNA and subsequently amplified through reverse transcription followed by amplification via polymerase chain reaction (RT/PCR).

Next, this DNA template is used to create an enriched RNA pool through in vitro transcription with a mutant T7 RNA polymerase that allows for the incorporation of 2′fluoro-modified pyrimidines. These modifications render the RNA more nuclease resistant. The steps leading to the creation of the enriched RNA pool are referred to as a “selection round”. The selection rounds against the target are typically continued until a plateau in binding affinity progression had been reached. Individual clones may then be isolated from the pool and sequenced.

RNA silencing moieties are well known in the art. For example, there are public databases from which one skilled in the art can choose one or more nucleic acid sequences that is an RNA silencing moiety targeting expression of a particular gene (as well as, in some cases, associated thermodynamic or biophysical properties), which can then be used in synthesizing a nucleic acid molecule useful with the methods of the present invention (see, e.g., on the Internet, such as, rnainterference.org; sirna.sbc.su.se; web.mit.edu/sirna; rnaiweb.com; and others). Thus, while illustrations of the invention described herein use either Plk1 or Bcl2 as gene targets, it is clear from the teachings herein that other RNA silencing moieties are readily available to be delivered using the invention to silence gene targets other than Plk1 and Bcl2. RNA silencing moieties having 2′ modifications at either or both of purines or pyrimidines, even at the Argonaute-2 cleavage site, have been shown to effect target gene knockdown at nanomolar concentrations. In one xample of the invention, an RNA silencing moiety comprises at least nucleotides that have a 2′-sugar modification. Commonly, for RNA silencing moieties comprising a sense strand and a separate antisense strand with each strand having one or more pyrimidine nucleotides and one or more purine nucleotides, 50 percent or more of the nucleotides in at least one strand of the dsRNA comprise a 2-sugar modification, wherein the 2′-sugar modification of any of the pyrimidine nucleotides differs from the 2′-sugar modification of any of the purine nucleotides (for example, a 2′fluoro modification, and a 2′methoxy modification, respectively). Further, the dsRNA either comprises zero single stranded nucleotide overhangs, or at least one single stranded nucleotide overhang, wherein each single stranded nucleotide overhang is six or fewer nucleotides in length.

### EXAMPLE 2

In this example, illustrated, described, and demonstrated are the following. Provided is a method of targeted delivery of an RNA silencing moiety, the method comprising contacting a nucleic acid molecule and cells in conditions effective, for the nucleic acid molecule to deliver the RNA silencing moiety into the cell cytoplasm such that the RNA silencing moiety is bound and processed by Dicer (i.e., is a Dicer substrate). This method involves the use of a nucleic acid molecule comprised of (i) a RNA silencing moiety, comprised of dsRNA comprising a guide strand to be delivered to Dicer; and (ii) and a targeting moiety for binding a surface receptor on a cell which, upon binding, results in cellular internalization to deliver the dsRNA to the cell cytoplasm to be accessible by Dicer, wherein the targeting moiety is an aptamer; and wherein the targeting moiety and at least one strand of the RNA silencing moiety are a contiguous nucleic acid molecule. Also provided is a method for making a Dicer substrate, comprising: (a) synthesizing a nucleic acid molecule comprising (i) a targeting moiety comprising an aptamer, and (ii) a first single stranded RNA (e.g., an RNA molecule comprising either a guide strand or a passenger strand) for forming a dsRNA, wherein the targeting moiety and the first single stranded RNA are a contiguous nucleic acid molecule; (b) hybridizing (annealing, base pairing) a second single stranded RNA, having full complementarity or partial complementarity to the first single stranded RNA, to the first single stranded RNA of the synthesized nucleic acid molecule in forming a dsRNA which can act as a Dicer substrate; wherein the targeting moiety is capable of binding to a receptor on the surface of a cell and subsequently being internalized into the cell, wherein the nucleic acid molecule is transported to the cell cytoplasm. Also provided is a method of introducing an RNA silencing moiety comprised of dsRNA into cells, the method comprising contacting cells with a nucleic acid molecule under conditions by which a targeting moiety of the nucleic acid molecule binds to a receptor on the cells, resulting in internalization of the nucleic acid molecule into the cells; wherein the nucleic acid molecule comprises a targeting moiety and a dsRNA molecule, wherein the targeting moiety and at least one strand of the dsRNA molecule are a contiguous nucleic acid molecule, wherein the targeting molecule is an aptamer that recognizes and binds to a cell surface receptor on the cells, and wherein a guide strand of the dsRNA becomes bound to Dicer subsequent to introduction of the nucleic acid molecule into the cells. Also provided is a method of RNA silencing (e.g., one or more of specific (based on sequence) gene knockdown, mRNA cleavage, or translational repression) resulting from such introduction into cells expressing the gene to be targeted.

In this example, the following sequences were used to illustrate the invention.

siRNAs

control siRNA (“CON”) target sequence:

| | |
|---|---|
| AATTCTCCGAACGTGTCACGT | (SEQ ID NO: 1) |

Plk1 siRNA target sequence:

| | |
|---|---|
| AAGGGCGGCTTTGCCAAGTGC | (SEQ ID NO: 2) |

Bcl-2 siRNA target sequence:

| | |
|---|---|
| NNGTGAAGTCAACATGCCTGC | (SEQ ID NO: 3) |

Dicer siRNA target sequence

NNCCTCACCAATGGGTCCTTT (SEQ ID NO: 4)

(where“N” is any of A, T, G or C)

### Nucleic Acid Sequences and Combinations

Aptamer A10:
(SEQ ID NO: 5)
5′GGGAGGACGAUGCGGAUCAGCCAUGUUUACGUCACUCCUUGUCAAU
CCUCAUCGGCAGACGACUCGCCCGA3′
“A10-CON”- Aptamer A-10 with CON Sense Strand
(the latter, in bold type):
(SEQ ID NO: 6)
5′GGGAGGACGAUGCGGAUCAGCCAUGUUUACGUCACUCCUUGUCAAU
CCUCAUCGGCAGACGACUCGCCCGAAAUUCUCCGAACGUGUCACGU3′
CON Antisense siRNA:
(SEQ ID NO: 7)
5′ACGUGACACGUUCGGAGAAdTdT3′
A10-Plk1 Sense Strand (the latter, in bold type):
(SEQ ID NO: 8)
5′GGGAGGACGAUGCGGAUCAGCCAUGUUUACGUCACUCCUUGUCAAU
CCUCAUCGGCAGACGACUCGCCCGAAAGGGCGGCUUUGCCAAGUGC3′
Plk1 Antisense siRNA:
(SEQ ID NO: 9)
5′GCACUUGGCAAAGCCGCCCdTdT3′
A10-Bc1-2 Sense Strand (the latter, in bold type):
(SEQ ID NO: 10)
5′GGGAGGACGAUGCGGAUCAGCCAUGUUUACGUCACUCCUUGUCAAU
CCUCAUCGGCAGACGACUCGCCCGAAAGUGAAGUCAACAUGCCUGC3′
Bcl-2 Antisense siRNA:
(SEQ ID NO: 11)
5′GCAGGCAUGUUGACUUCACUU-3′
MutA10 (point mutations “CC” in A10)-Plk1
Sense Strand (the latter, in bold type):
(SEQ ID NO: 12)
5′GGGAGGACGAUGCGGAUCAGCCAUCCUUACGUCACUCCUUGUCAAU
CCUCAUCGGCAGACGACUCGCCCGAAAGGGCGGCUUUGCCAAGUGC3′
A10 5′-primer:
(SEQ ID NO: 13)
5′TAATACGACTCACTATAGGGAGGACGATGCGG3′
A10 3′-primer:
(SEQ ID NO: 14)
5′TCGGGCGAGTCGTCTG3′
A10 template primer:
(SEQ ID NO: 15)
5′GGGAGGACGATGCGGATCAGCCATGTTTACGTCACTCCTTGTCAAT
CCTCATCGGCAGACGACTCGCCCGA3′
Control siRNA 3′-primer:
(SEQ ID NO: 16)
5′ACGTGACACGTTCGGAGAATTTCGGGCGAGTCGTCTG3′
Plk1 siRNA 3′-primer:
(SEQ ID NO: 17)
5′GCACTTGGCAAAGCCGCCCTTTCGGGCGAGTCGTCTG3′
Bcl-2 siRNA 3′-primer:
(SEQ ID NO: 18)
5′GCAGGCATGTTGACTTCACTTTCGGGCGAGTCGTCTG3′
A10 mutant primer:
(SEQ ID NO: 19)
5′AGGACGATGCGGATCAGCCATCCTTACGTCA3′

### Nucleic Acid Molecules

A nucleic acid molecule comprising a targeting moiety (in this Example, aptamer A10; SEQ ID NO:5) and a dsRNA molecule (in this Example, targeting either Plk1 (e.g., formed by annealing SEQ ID NO:2 with SEQ ID NO:9), or Bcl2 (e.g., formed by annealing SEQ ID NO:3 with SEQ ID NO:11), wherein the targeting moiety and at least one strand of the dsRNA molecule are a contiguous nucleic acid molecule (see, e.g., SEQ ID NO:8 or SEQ ID NO:10), were generated in order to specifically target siRNAs to cells expressing the cell-surface receptor PSMA. The aptamer portion of the chimera (A10) mediates binding to PSMA and subsequent internalization. The siRNA portion targets the expression of survival genes such as Plk1 or Bcl2, and could be engineered to include both. Also produced, for experimental controls, were A10-CON (A10 and a non-silencing siRNA, SEQ ID NO:6); and mutA10-Plk1 (SEQ ID NO:12), wherein mutA10 contains two point mutations in A10 region which disrupt the secondary structure of the putative PSMA-binding portion of the A10 aptamer. Thus, one representative nucleic acid molecule comprising a targeting moiety and an RNA silencing moiety is illustrated by a chimera in which the nucleic acid sequence of SEQ ID NO:8 is hybridized to a nucleic acid sequence of SEQ ID NO:9 (in forming “A10-Plk1 chimera”). Another representative nucleic acid molecule comprising a targeting moiety and an RNA silencing moiety is illustrated by a chimera in which the nucleic acid sequence of SEQ ID NO:10 is hybridized to a nucleic acid sequence of SEQ ID NO:11 (in forming “A10-Bcl2 chimera”).

Double-stranded DNA templates were generated by polymerase chain reaction (“PCR”) as follows. The A10 template primer (SEQ ID NO:15) was used as a template for the PCRs with the A10 5′-primer and one of the following 3′-primers: A10 3′-primer (for the A10 aptamer; SEQ ID NO:14), Control siRNA 3′-primer (for the A10-CON chimera; SEQ ID NO:16), Plk1 siRNA 3′-primer (for the A10-Plk1 chimera; SEQ ID NO:17) or Bcl-2 siRNA 3′-primer (for the A10-Bcl-2 chimera; SEQ ID NO:18). Templates for transcription were generated in this way or by cloning these PCR products into a T-A cloning vector, and using the clones as templates for PCR with the appropriate primers. The DNA encoding the mutA10-Plk1 chimera was prepared by sequential PCRs. In the first reaction, the A10 template primer was used as the template with the A10 mutant primer as the 5′-primer and the Plk1 siRNA 3′-primer as the 3′-primer. The product of this reaction was purified and used as the template for a second reaction with the A10 5′-primer (SEQ ID NO:13) and the Plk1 siRNA 3′-primer. The resulting PCR product was cloned into the cloning vector, and sequenced. This clone was used as the template in a PCR with the A10 5′-primer and the Plk-1 3′-primer to generate the template for transcription. Fluorescent aptamer and aptamer-siRNA chimeras were in vitro transcribed in the presence of (5′-FAM) (spacer 9)-G-3′ (FAM-labeled G), wherein FAM is carboxyfluorescein, as described below.

Transcriptions were set up either with or without 4 mM FAM-labeled G. For a 250 μL transcription reactions: 50 μL 5× T7 RNAP Buffer optimized for 2′F transcriptions (20% w/v PEG 8000, 200 mM Tris-HCl pH 8.0, 60 mM MgCl₂, 5 mM spermidine HCl, 0.01% w/v triton X-100, 25 mM DTT (dithiothreitol)), 25 μL 10× 2′F-dNTPs (2′fluoro-dinucleotriphosphates; 30 mM 2′F-CTP, 30 mM 2′F-UTP, 10 mM 2′OH-ATP, 10 mM 2′ OH-GTP), 2 μL IPPI, 300 pmoles aptamer-siRNA chimera PCR template, 3 μL T7 (Y639F) polymerase, bring up to 250 μL with milliQ H₂O.

### A10 Aptamer-siRNA Chimeras Bind Specifically to PSMA-Expressing Cells.

First, the ability of the A10 aptamer-siRNA chimeras to bind the surface of cells expressing PSMA was tested. Previously, PSMA has been shown to be expressed on the surface of LNCaP prostate carcinoma cell line (ATCC#CRL-1740), but not the surface of PC-3 prostate carcinoma cell line (ATCC#CRL-1435). To determine whether the A10 aptamer-siRNA chimeras can bind the surface of cells expressing PSMA, fluorescently labeled A10 (SEQ ID NO:5), A10-CON chimera (SEQ ID NO:6 annealed to SEQ ID NO:7), or A10-Plk1 chimera were incubated with either LNCaP or PC-3 cells. PC-3 or LNCaP cells were trypsinized, washed twice with 500 μL PBS, and fixed in 400 μL of FIX solution (PBS+1% formaldehyde) for 20 minutes at room temperature. After washing cells to remove any residual trace of formaldehyde, cell pellets were resuspended in 1× Binding Buffer (1×BB) (20 mM HEPES pH 7.4, 150 mM NaCl, 2 mM CaCl₂, 0.01% BSA) and incubated at 37° C. for 20 minutes. Cells were then pelleted and resuspended in 50 μL of 1×BB (pre-warmed at 37° C.) containing either 400 nM FAM-G labeled A10 aptamer or 400 nM FAM-G-labeled aptamer-siRNA chimeras. Due to the low incorporation efficiency of FAM-G during the transcription reaction, for comparison of A10-Plk1 chimera and mutA10-Plk1 chimera cell surface binding up to 10 μM of FAM-G labeled aptamer chimeras were used. Concentrations of FAM-G labeled aptamer and aptamer-siRNA chimeras for the relative affinity measurements varied from 0 to 4 μM. Cells were incubated with the RNA for 40 minutes at 37° C., washed three times with 500 μL of 1×BB pre-warmed at 37° C., and finally resuspended in 400 μL of FIX solution pre-warmed at 37° C. Cells were then assayed using flow cytometry as detailed above and the relative cell surface binding affinities of the A10 aptamer and A10 aptamer-siRNA chimera derivatives were determined.

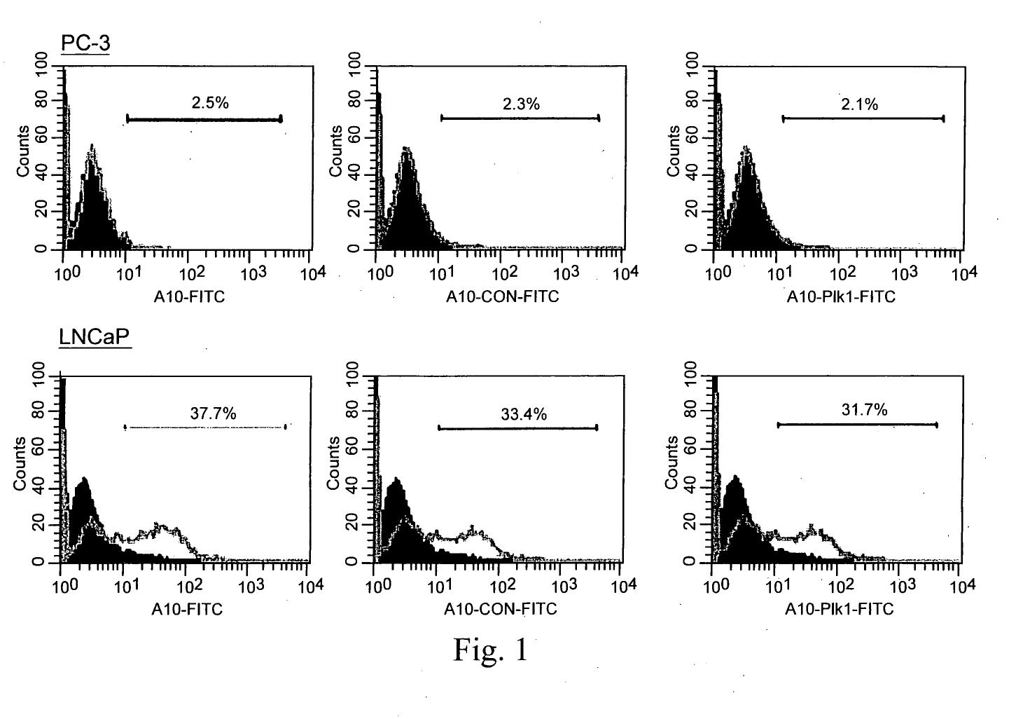

As shown in FIG. 1, binding of A10 aptamer and A10 aptamer-siRNA chimeras was specific to LNCaP cells, as binding was undetectable in PC-3 cells. Binding to LNCaP cells was dependent on the region of A10 aptamer predicted to bind PSMA, as the mutA10-Plk1 chimera was unable to bind to LNCaP cells in flow cytometric analyses. Also shown by FIG. 1, the aptamer-siRNA chimera and the A10 aptamer were found to bind to the surface of LNCaP cells with comparable affinities. To verify that the A10 aptamer-siRNA chimeras were indeed binding to PSMA, LNCaP cells were incubated with (1 μM) of either fluorescently labeled A10, A10-CON, or A10-Plk1 RNA and competed with increasing amounts (from 0 μM to 4 μM) of unlabled A10 aptamer or with an antibody specific for human PSMA (“competitors”). Bound fluorescently labeled RNAs in the presence of increasing amounts of a competitor were assessed using flow cytometry. Binding of the labeled A10 aptamer and A10 aptamer-siRNA chimeras (A10-CON, and A10-Plk1) to LNCaP cells was equally competed with either unlabeled A10 aptamer or the anti-PSMA antibody indicating that these RNAs are binding PSMA on the surface of LNCaP cells.

### Aptamer-siRNA Chimeras Specifically Silence Gene Expression.

To determine whether the aptamer-siRNA chimeras can silence target gene expression, A10 aptamer-siRNA chimeras were assayed for their ability to deliver siRNAs against Plk1 or Bcl2 to cells in cell culture (37° C., and 5% CO₂ in RPMI 1640 (LNCaP cells) or Ham's F12-K medium (PC-3 cells), supplemented with 10% fetal bovine serum). For A10 aptamer and A10 aptamer-siRNA chimeras, PC-3 or LNCaP cells were seeded in 6-well plates at 60% confluency. In these cell cultures, cells were treated by contacting the cells with either 400 nM of A10 aptamer or of A10 aptamer-siRNA chimera on days 2 and 4. Cells were collected from the cultures on day 5 for analysis.

Gene silencing was assessed by flow cytometry or immunoblotting using antibodies specific to either human PLK1 or human BCL-2, as compared to levels of silencing achieved by transfecting the respective siRNAs. For siRNA transfections, PC-3 or LNCaP cells were seeded in 6-well plates at 60% confluency. Cells were transfected with either 200 nM or 400 nM siRNA on days 2 and 4 using a commercial transfection reagent and following the manufacturer's recommendations. Cells were collected on day 5 for analysis. For flow cytometric analyses, PC-3 or LNCaP cells were trypsinized, washed three times in phosphate buffered saline (PBS), and counted using a hemocytometer. 200,000 cells (5×10⁵ cells/mL) were resuspended in 400 μl of PERM/FIX buffer, and incubated at room temperature (RT) for 20 minutes. Cells were then pelleted and washed three times with 1× PERM/WASH buffer. Cells were then resuspended in 50 μL 1× PERM/WASH buffer containing 20 μg/mL of primary antibody against either human PLK1, or human BCL-2, or 20 μg/mL of isotype-specific control antibody. After 40 minutes of incubation at RT, cells were washed three times with 500 μL 1× PERM/WASH buffer and incubated with a 1:500 dilution of secondary antibody (anti-mouse IgG-APC) in 1× PERM/WASH for 30 minutes at RT. Cells were washed as detailed above and analyzed by flow cytometry. For analyses by immunoblotting, LNCaP cells were transfected with control siRNA, or siRNAs to either Plk1 or Bcl-2 as described above. Cells were trypsinized, washed in PBS, and cell pellets were resuspended in 1× RIPA buffer and incubated on ice for 20 minutes. Cells were then pelleted and 50 μg of total protein from the supernatants were resolved on either 8.5% SDS-PAGE gel for Plk1 or a 15% SDS-PAGE gel for Bcl-2. Plk1 gene expression was detected using a commercially available antibody specific to human PLK1. Bcl-2 gene expression was detected using a commercially available antibody specific to human BCL-2.

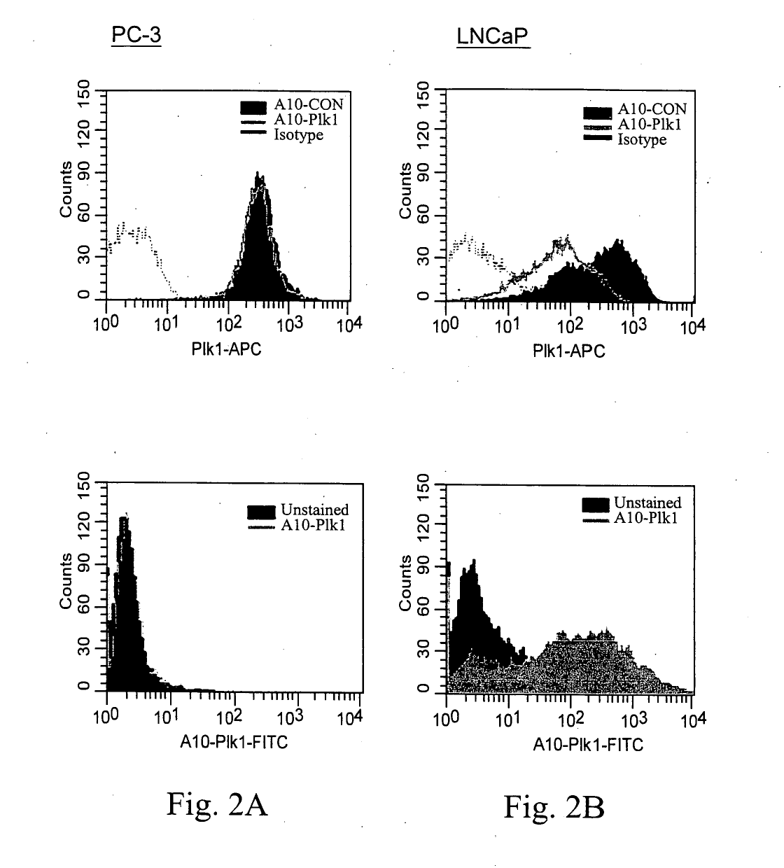

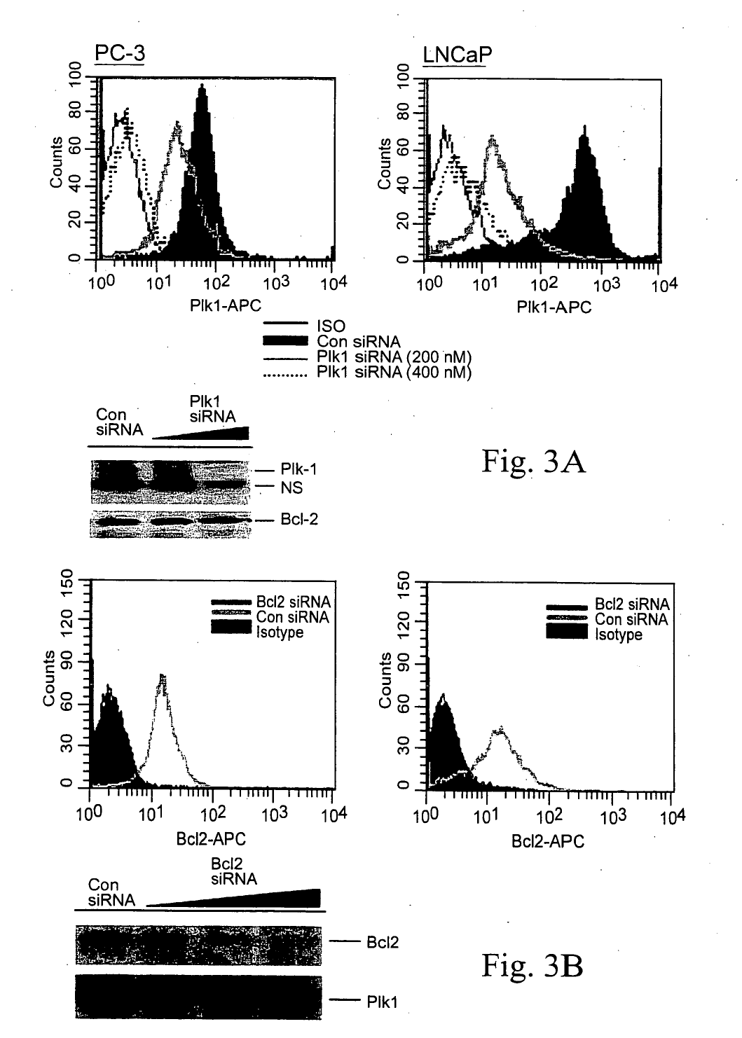

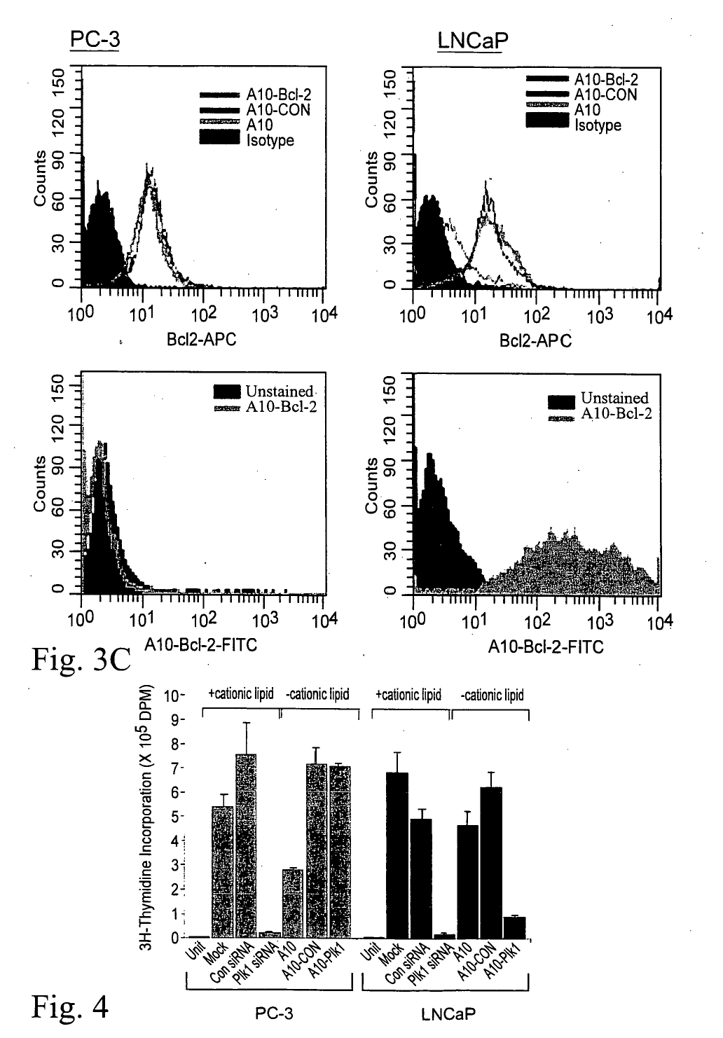

As shown in FIG. 2A, PLK1 is detected in PC-3 cells treated with either A10-Plk1 chimera or A10-CON chimera. The A10-Plk1 chimera fails to knockdown Plk1 expression in the PSMA-negative PC-3 cells, and no uptake of the A10-Plk1 chimera (FITC-labeled) was detected in PC-3 cells. As compared to the level of Plk1 silencing achieved with transfection of LNCaP cells using Plk1 siRNA in transfection reagent (FIG. 3A), or to the level of Plk1 expressed in LNCaP cells treated with the experimental control (A10-CON chimera, with CON as a non-silencing RNA; FIG. 2B), treatment of LNCaP cells with A10-Plk1 chimera results in decreased detection (e.g., silencing) of Plk1 gene expression, and binding to/uptake of fluorescent-labeled (FITC) A10-Plk1 chimera aptamer into LNCaP cells.

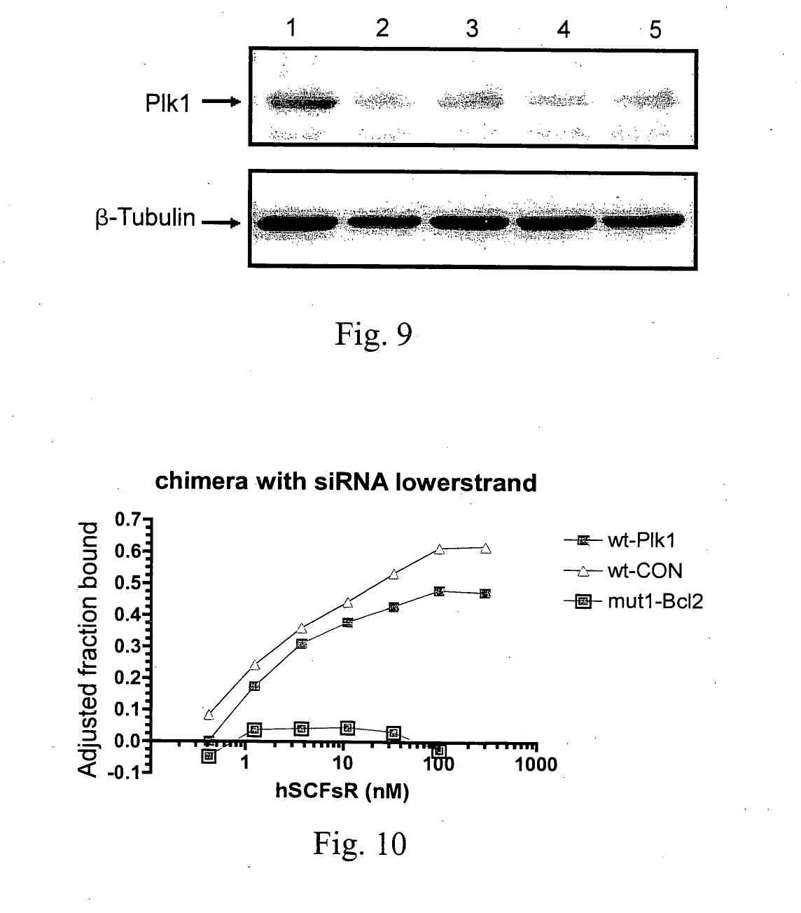

As shown in FIG. 3C, BCL2 is detected in PC-3 cells treated with either A10-Bcl2 chimera or A10-CON chimera. The A10-Bcl2 chimera fails to knockdown Bcl2 expression in the PSMA-negative PC-3 cells, and no uptake of the A10-Bcl2 chimera (FITC-labeled) was detected in PC-3 cells. As compared to the level of Bcl2 silencing achieved with transfection of LNCaP cells using Bcl2 siRNA in transfection reagent (FIG. 9B), or to the level of Bcl2 expressed in LNCaP cells treated with the experimental control (A10-CON chimera, with CON as a non-silencing RNA; FIG. 3C), treatment of LNCaP cells with A10-Bcl2 chimera results in decreased detection (silencing) of Bcl2 expression, and binding to/uptake of fluorescent-labeled (FITC) A10-Bcl2 chimera into LNCaP cells.

Thus, these results show that a nucleic acid molecule comprising a targeting moiety (an aptamer) and a dsRNA molecule (siRNA), wherein the targeting moiety and at least one strand of the dsRNA molecule are a contiguous nucleic acid molecule, can be contacted with the target cells, and internalized into targeted cells. Inside the targeted cells, the siRNA is delivered to the siRNA processing machinery in the cytoplasm (e.g., Dicer) such that gene silencing is effected.

LNCaP cells pre-treated with 5-α-dihydrotestosterone (DHT) has been shown to reduce the expression of PSMA. Treatment of LNCaP cells with 2 nM DHT for 48 hours greatly reduced the expression of PSMA such that cell surface expression of PSMA was reduced from 73.2% to 13.4% as determined by flow cytometry. To further verify that silencing by A10 aptamer-siRNA chimeras was indeed dependent on PSMA, LNCaP cells were incubated with or without 2 nM DHT for 48 hours prior to addition of A10-Plk1 chimera. Uptake of A10-Plk1 chimera into cells, and silencing of Plk1 gene expression observed in LNCaP cells treated with the A10-Plk1 chimera, were substantially decreased in LNCaP cells treated with DHT. These data, together with the cell surface binding data, indicate that cell-type specific silencing is dependent upon cell surface expression of PSMA; i.e., that the targeting moiety delivers the dsRNA to and into the cell for delivery of the siRNA to the siRNA processing machinery. As expected, mutA10-Plk1 did not bind to the surface of LNCaP cells either in the presence or absence of DHT treatment.

### Knockdown of Gene Expression by Aptamer-siRNA Chimera Results in Functional Changes of Cells Expressing PSMA

As another indication of gene silencing mediated by a method according to the present invention, assayed was the ability of an aptamer-siRNA chimera (wherein the siRNA targets oncogenes or anti-apoptotic genes) to reduce cell proliferation and induce apoptosis. For assaying cell proliferation, PC-3 cells or LNCaP cells were treated with A10-CON or A10-Plk1 aptamer-siRNA chimeras and cell proliferation was measured by ³H-thymidine incorporation. PC-3 and LNCaP cells previously treated with siRNAs (CON siRNA (SEQ ID NO:1 annealed with SEQ ID NO;7) or Plk1 siRNA with cationic lipid transfection reagent) or aptamer-siRNA chimeras (absent transfection reagent) as detailed above, were trypsinized and seeded in 12-well plates at ˜20,000 cells/well. Cells were then forced into a G1/S block by addition of 0.5 μM hydroxy urea (HU). After 21 hours, cells were released from the HU block by addition of media lacking HU, and then incubated with media containing ³H-thymidine (1 μCi/mL medium) to monitor DNA synthesis. After a 24 hour incubation in the presence of media containing ³H-thymidine, cells were washed twice with PBS, washed once with 5% w/v trichloroacetic acid (TCA) (VWR), collected in 0.5 mL of 0.5N NaOH (VWR) and placed in scintillation vials for measurement of ³H-thymidine incorporation. As shown in FIG. 4, in LNCaP cells, proliferation was effectively reduced by the A10-Plk1 chimera, but not by the control A10-CON chimera. This effect was specific for cells expressing PSMA, as it was not seen in the PC-3 cells. Treatment of LNCaP cells with the A10-Plk1 chimera reduced proliferation to nearly the same extent as observed when cationic lipids were employed to transfect Plk1 siRNA, even though no transfection reagent was utilized for aptamer-siRNA chimera delivery (FIG. 4).

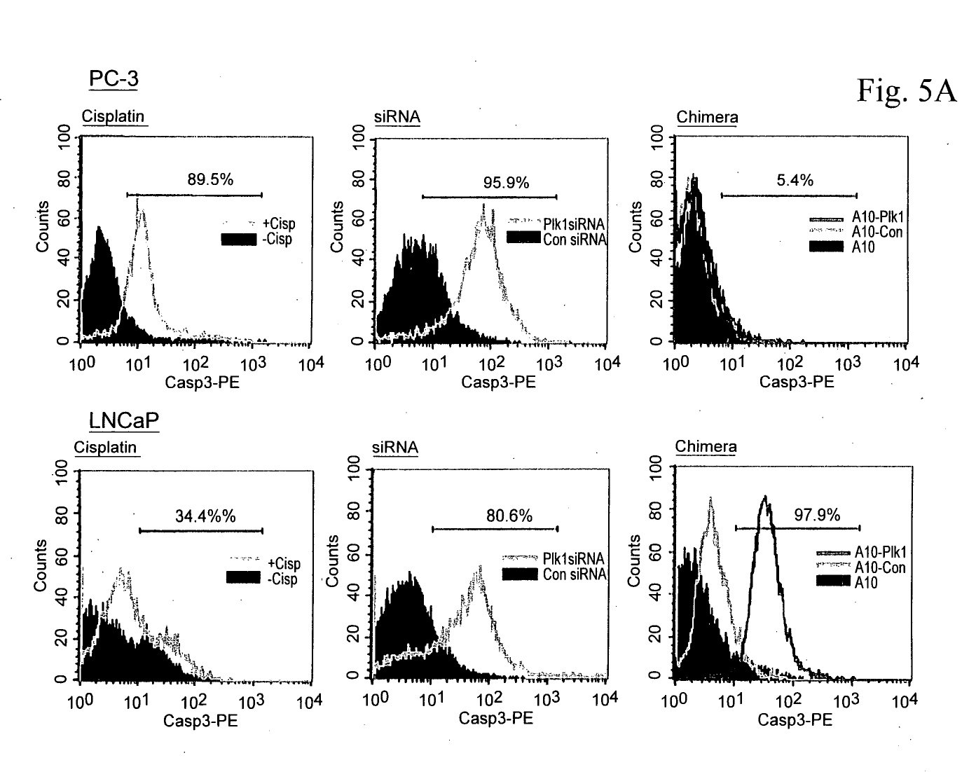

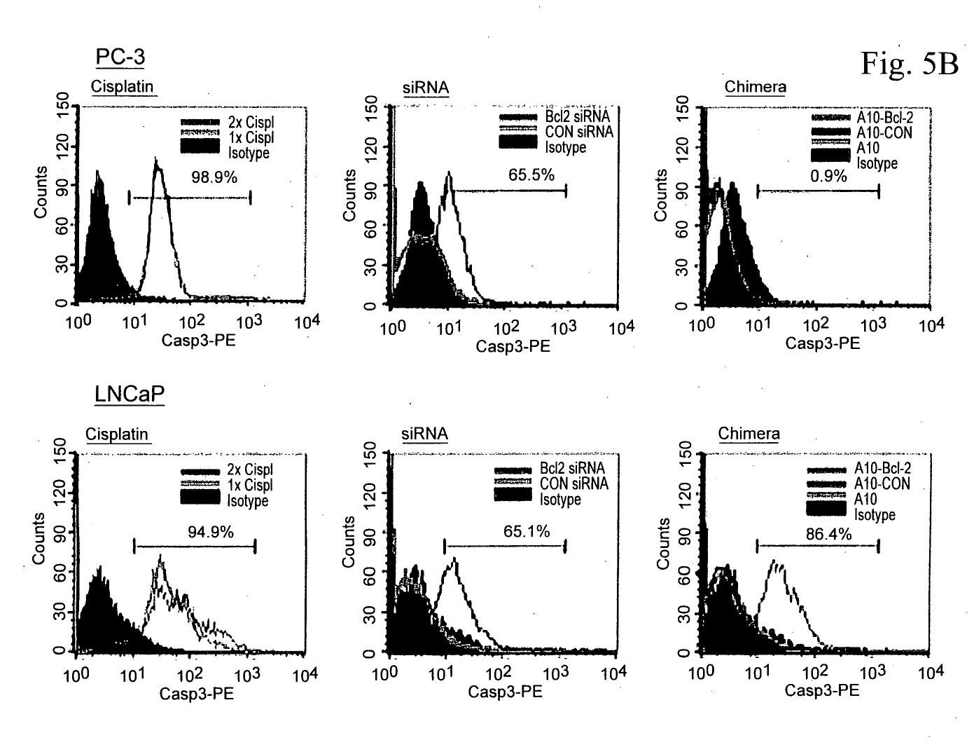

Next, the ability of the A10-Plk1 and A10-Bcl-2 chimeras to induce apoptosis of prostate cancer cells expressing PSMA was assessed using a standard assay for measuring production of caspase 3, induction of which is a known indicator of apoptosis. PC-3 cells or LNCaP cells were either treated by addition of A10 aptamer, A10-CON chiemra, A10 Plk1 chimera, or A10-Bcl2 chimera, to the media; or the cells were transfected with siRNAs to Plk1 or Bcl2 using cationic lipids; as described above. Apoptosis was assessed by measuring production of active caspase 3 (Casp3) by flow cytometry. While transfected siRNAs to Plk1 and Bcl-2 induced apoptosis of both PC-3 and LNCaP cells, apoptosis induced by the aptamer-siRNA chimeras was specific to LNCaP cells and did not require a transfection reagent. Treatment of PC-3 and LNCaP cells with cisplatin was used as a positive control for apoptosis (cells treated with medium containing 100 μM (1×) or 200 μM (2×) cisplatin for 30 hours). Cells were then fixed and stained for active caspase 3 using a commercially available PE-conjugated antibody specific to cleaved caspase 3, as specified in manufacturer's protocol. Flow cytometric analysis was used to quantitate % PE-positive cells as a measure of apoptosis. As shown in FIG. 5 (FIG. 5A for Plk1; FIG. 5B for Bcl2), while transfected siRNAs to Plk1 and Bcl-2 induced apoptosis of both PC-3 cells and LNCaP cells, apoptosis induced by the aptamer-siRNA chimeras A10-PlK1 or A10-Bcl2 was specific to LNCaP cells. Treatment of LNCaP cells with the A10-Plk1 chimera or A10-Bcl2 chimera (in the absence of a transfection reagent) induced apoptosis to nearly the same extent as observed when cationic lipids were employed to transfect Plk1 siRNA or Bcl2 siRNA.

These results are further evidence that using a method of delivery according to the present invention, target cells (having a cell surface receptor recognized by the aptamer portion) can be contacted with nucleic acid molecule comprising a targeting moiety (an aptamer) and an RNA silencing moiety comprising dsRNA (e.g., siRNA), resulting in internalization of the nucleic acid molecule into targeted cells, and delivery of the RNA silencing moiety to the siRNA processing machinery in the cytoplasm (e.g., Dicer) such that gene silencing is effected. Gene silencing can be measured not only by a reduction in the amount of the gene product (e.g., protein) produced, but also by measuring a known cell function that results from silencing of the gene targeted by the dsRNA portion of the nucleic acid molecule (e.g., cell proliferation, cell replication, cell apoptosis, cytokine production, cell differentiation, enhanced cell survival, upregulation of an immune response (e.g., an anti-tumor response, and antiviral response), etc., depending on the gene targeted).

### The Nucleic Acid Molecule as a Dicer Substrate

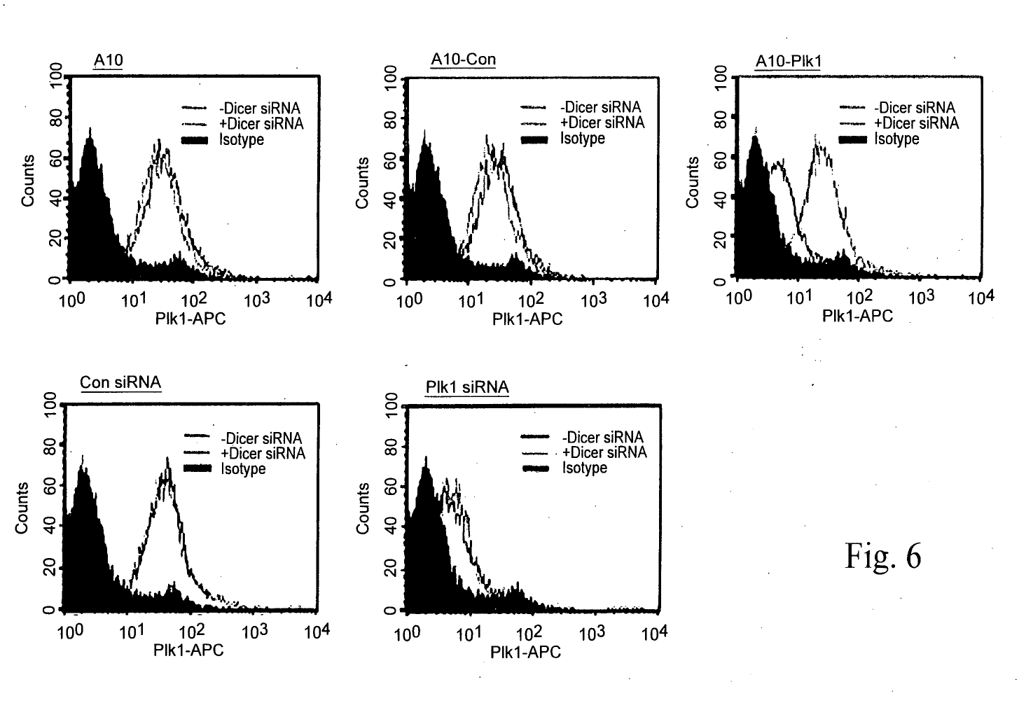

To show that the mechanism by which aptamer-siRNA chimeras silence gene expression is dependent on Dicer activity, silencing activity was assessed in the presence or absence of Dicer. Therefore, the Dicer protein level was reduced by targeting its expression with an known siRNA against human Dicer (SEQ ID NO:4). LNCaP cells were seeded in 6-well plates at 200,000 cells per well. After 24 hours, cells were co-transfected with either 400 nM of control siRNA, 400 nM of Plk1 siRNA, 400 nM A10 aptamer, or 400 nM of A10 aptamer-siRNA chimeras alone, in the presence or absence of an siRNA to human Dicer, as described above and using a transfection reagent. Cells were then collected and processed for flow cytometric analysis using an antibody specific for human Plk1, as described above. As shown in FIG. 6, silencing of Plk1 gene expression by the A10-Plk1 chimera was inhibited by co-transfection of Dicer siRNA (FIG. 6, top row, right panel) suggesting that aptamer-siRNA chimera-mediated gene silencing is dependent on Dicer and occurs via the RNAi pathway. In contrast, as expected, inhibition of Dicer had no effect on Plk1 siRNA-mediated silencing (FIG. 6, bottom, right panel) because siRNAs of 21-23 nt in length have been shown to by-pass the Dicer step.

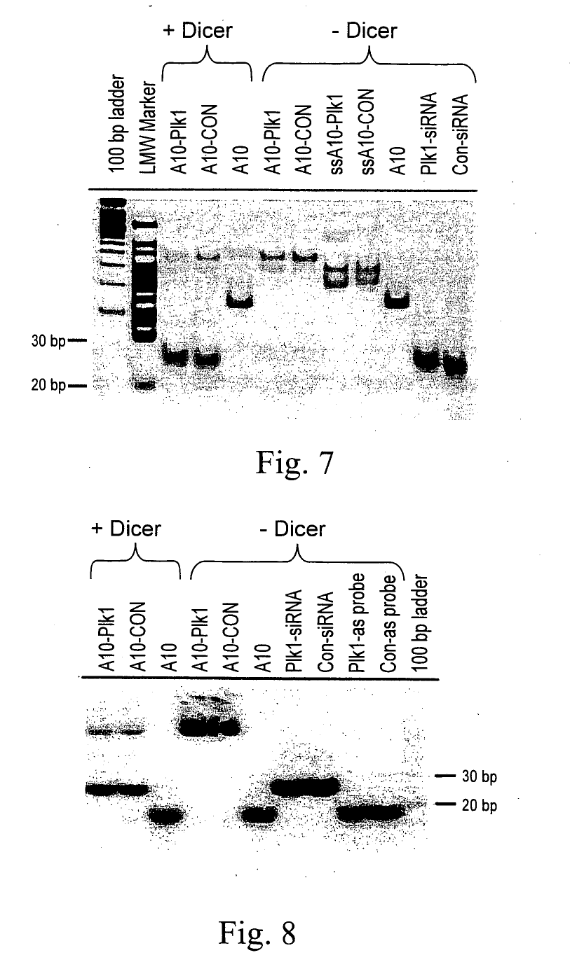

To test whether the aptamer-siRNA chimeras were directly cleaved by Dicer to produce 21-23 nt siRNA fragments corresponding to the siRNA sequences engineered in the chimeric constructs, a Dicer digestion assay was used. 1-2 μg of A10 aptamer or A10 aptamer-siRNA chimeras were digested using a kit with a recombinant dicer enzyme and following the manufacturer's recommendations. Digests were then resolved by polyacrylamide gel electrophoresis (PAGE) on a 15% non-denaturing polyacrylamide gel, and stained with ethidium bromide prior to visualization. Alternatively, 1-2 μg of A10 aptamer or A10 aptamer-siRNA chimera sense strands (ssA10-Plk1 (SEQ ID NO:8), ssA10-CON (SEQ ID NO:6), without the complementary antisense siRNA strand) were annealed to ³²P-end-labeled complementary antisense siRNAs (probe). The siRNAs were end-labeled using T4 polynucleotide kinase following the manufacturer's recommendations. The antisense siRNA were not complementary to the A10 aptamer. A10 aptamer or the annealed chimeras (A10-CON chimera or A10-Plk1 chimera) were incubated with or without Dicer enzyme and subsequently resolved on a 15% non-denaturing PAGE gel as described above. The gel was dried and exposed to radiographic film for 5 minutes. As shown in FIG. 7, the aptamer-siRNA chimeras (“A10-CON” or “A10-Plk1”), but not A10 aptamer (“A10”) or the longer single-stranded sense strand of the aptamer-siRNA chimeras (“ssA10-CON” or “ssA10-Plk1”), was digested by Dicer enzyme to release 21-23 nt fragments in length. To verify that these 21-23 nt long Dicer fragments correspond to the control and Plk1 siRNAs, the A10-aptamer-siRNA chimeras were labeled by annealing the complementary ³²P-end labeled anti-sense strand of the siRNAs and incubated with or without recombinant Dicer (FIG. 8). As shown in FIG. 8, digest of labeled A10-CON or A10-Plk1 with recombinant Dicer resulted in release of 21-23 nt long fragments that retained the ³²P-end labeled anti-sense strand indicating that these fragments are indeed the siRNA portion of the aptamer-siRNA chimeras.

These results are further evidence that the nucleic acid molecule delivered by the method of the present invention can reach Dicer such that the dsRNA is a substrate for Dicer cleavage. Dicer was also found to cleave the double-stranded, dsRNA portion of the nucleic acid molecule from the aptamer portion of the nucleic acid molecule, a step that would be expected to precede incorporation of the shorter strand of the RNAi into RISC complexes. Thus, RNAi observed using the methods of the present invention result from cell-type specific introduction of dsRNA into the siRNA processing pathways to yield functional siRNA (e.g., functional in that there is subsequent RNAi).

### Aptamer-siRNA Chimeras Do Not Trigger Interferon Responses.

Various groups have reported that delivered siRNAs can potentially activate non-specific inflammatory responses, leading to cellular toxicity. Therefore, a determination was made of the amount of interferon-beta (INF-β) produced by PC-3 cells or LNCaP cells that were either untreated, transfected with siRNAs to Plk1 or Bcl-2, or treated with the aptamer-siRNA chimeras (in the absence of transfection reagents). To assay for the presence or absence of secreted IFN-β from treated and untreated PC-3 and LNCaP cells, used was a human Interferon beta ELISA kit and followed was the kit manufacturer's recommendations. Briefly, cells were seeded at 200,000 cells/well in 6 well plates. Twenty-four hours later, cells were either transfected with a mixture of transfection reagent plus varying amounts of Poly(I:C) (2.5, 5, 10, 15 μg/ml) as a positive control for INF-β, or with a mixture of transfection reagent and either CON siRNA or siRNAs to Plk1 or Bcl2 (200 nm or 400 nm). In addition, cells were treated with 400 nM each of A10 aptamer and A10 aptamer-siRNA chimeras as described above. Forty eight hours after the various treatments, 100 μL of supernatant from each treatment group was added to a well of a 96-well plate and incubated at room temperature for 24 hours. Presence or absence of INF-β in the supernatants was detected using an antibody specific to human INF-β, and following the manufacturer's recommendations. Unlike treatment of the cells with Poly(I:C) (the positive control for inducing INF-β) which resulted in significant production of INF-β, treatment with either siRNAs or aptamer-siRNA chimeras did not induce production of INF-β (above background levels of INF-β detected in this assay) under these experimental conditions. These results suggest that delivery of aptamer-siRNA chimeras to cells does not trigger a substantial interferon response.

A10-Plk1 Mediates Tumor Regression in a Standard Experimental Model of Human Prostate cancer.

The efficiency and specificity of the A10-Plk1 chimera in a bearing tumors derived from either PSMA-positive human prostate cancer cells (LNCaP) or PSMA-negative human prostate cancer cells (PC-3) was also evaluated in an experimental model of human prostate cancer. Athymic mice were inoculated with either 5×10⁶ (in 100 μl of 50% matrigel) in vitro propagated PC-3 cells or LNCaP cells subcutaneously injected into each flank. Approximately, thirty-two non-necrotic tumors for each tumor type which exceeded 1 cm in diameter were randomly divided into four groups of eight mice per treatment group as follows: group 1, no treatment (DPBS); group 2, treated with A10-CON chimera (200 pmols/injection×10); group 3, treated with A10-Plk1 chimera (200 pmols/injection×10); group 4, treated with mut-A10-Plk1 chimera (200 pmols/injection×10). Compounds were injected intratumorally in 75 μL volumes every other day for a total of 20 days. Day 0 marks the first day of injection. The small volume injections are small enough to preclude the compounds being forced inside the cells due to a non-specific high-pressure injection. Tumors were measured every three days with calipers in three dimensions. The following formula was used to calculate tumor volume: V_{T}=(WXLXH)×0.5236 (W, the shortest dimension; L, the longest dimension). The growth curves are plotted as the means tumor volume±SEM. The experiment was terminated by euthanasia 3 days after the last treatment when the tumors were excised and formalin fixed for immunohistochemistry.

No difference in tumor volume was observed with the PC-3 tumors with any of the different treatments indicating that the chimeric RNAs did not have any non-specific cell killing effect. In contrast, a pronounced reduction in tumor volume was observed for LNCaP tumors treated with A10-Plk1 chimera. Indeed, from Day 6 to Day 21 the various control treated tumors (group 1, group 2, group 4) increased 3.63 fold in volume (n=22) while the A10-Plk1 chimera-treated group (group 3) had a 2.21 fold reduction in volume (n=8). Regression of LNCaP tumor volume in this experimental model was specific to treatment with the A10-Plk1 chimera, and was not observed with DPBS treatment or treatment with the A10-CON or mutA10-Plk1 chimeric RNAs. Importantly, no morbidity or mortality was observed in treatment group 3 following the 20-day treatment with the chimeric RNAs suggesting that these compounds are not toxic to the animals under the conditions of these experiments. It is noteworthy that the RNA used to make the nucleic acid molecules described herein (targeting moiety and an RNA silencing moiety) are protected from rapid degradation by extracellular RNAses by the 2′-fluoro modification of the pyrimidines in the aptamer sense strand, which likely contribute to their performance in vivo (as well as in vitro in the presence of serum).

### EXAMPLE 3

This is another illustrative example describing and demonstrating the invention. Provided is a method of targeted delivery of an RNA silencing moiety, the method comprising contacting a nucleic acid molecule and cells in conditions effective for the nucleic acid molecule to deliver the RNA silencing moiety into the cell cytoplasm such that the RNA silencing moiety is bound and processed by Dicer (i.e., is a Dicer substrate). This method involves the use of a nucleic acid molecule composed of (i) a RNA silencing moiety, comprised of dsRNA comprising a guide strand to be delivered to Dicer; and (ii) and a targeting moiety for binding a receptor on a cell, wherein the targeting moiety is an aptamer; wherein the targeting moiety and at least one strand of the RNA silencing moiety are a contiguous nucleic acid molecule; and wherein binding of the target moiety to the receptor results in the nucleic acid molecule being internalized into the cell to deliver the dsRNA to the cell cytoplasm to be accessible by Dicer. Also provided is a method for making a Dicer substrate, comprising: (a) synthesizing a nucleic acid molecule comprising (i) a targeting moiety comprising an aptamer, and (ii) a first single stranded RNA (e.g., an RNA molecule comprising either a guide strand or a passenger strand) for forming a dsRNA; wherein the targeting moiety and the first single stranded RNA are a contiguous nucleic acid molecule; (b) hybridizing a second single stranded RNA, having full complementarity or partial complementarity to the first single stranded RNA, to the first single stranded RNA of the synthesized RNA molecule in forming a dsRNA which can act as a Dicer substrate; and wherein the targeting moiety is capable of binding to a receptor on the surface of a cell with subsequent internalization of the nucleic acid molecule into the cell in delivering the nucleic acid molecule to the cell cytoplasm so that the dsRNA portion is accessible to Dicer and becomes a Dicer substrate. Also provided is a method of introducing an RNA silencing moiety comprised of dsRNA into cells, the method comprising contacting cells with a nucleic acid molecule under conditions by which a targeting moiety of the nucleic acid molecule binds to a receptor on the cells, resulting in internalization of the nucleic acid molecule into the cells; wherein the nucleic acid molecule comprises a targeting moiety and a dsRNA molecule, wherein the targeting moiety and at least one strand of the dsRNA molecule are a contiguous nucleic acid molecule, wherein the targeting molecule is an aptamer that recognizes a cell surface receptor on the cells, and wherein a guide strand of the dsRNA becomes bound to Dicer subsequent to introduction of the nucleic acid molecule into the cells.

In this example, the following sequences were used to illustrate the invention.

Plk1 siRNA target sequence:
(SEQ ID NO: 2)
AAGGGCGGCTTTGCCAAGTGC
A9-Plk1 Sense Strand (the latter, in bold type):
(SEQ ID NO: 20)
5′GGGAGGACGAUGCGGACCGAAAAAGACCUGACUUCUAUACUAAGUC
UACGUUCCCAGACGACUCGCCCGAAAGGGCGGCUUUGCCAAGUGC3′
Plk1 Antisense siRNA:
(SEQ ID NO: 9)
5′GCACUUGGCAAAGCCGCCCdTdT3′

### Nucleic Acid Molecules

A nucleic acid molecule comprising a targeting moiety (in this Example, aptamer A9) and a dsRNA molecule (in this Example, targeting Plk1), wherein the targeting moiety and at least one strand of the dsRNA molecule are a contiguous nucleic acid molecule (e.g., see SEQ ID NO:20), were generated in order to specifically target siRNAs to cells expressing the cell-surface receptor PSMA. The aptamer portion of the chimera (A9) mediates binding to PSMA and subsequent internalization. The siRNA portion targets the expression of survival gene, Plk1. Thus, one representative nucleic acid molecule comprising a targeting moiety and an RNA silencing moiety is illustrated by a molecule in which the nucleic acid sequence of SEQ ID NO:20 is hybridized to a nucleic acid sequence of SEQ ID NO:9 (in forming “A9-Plk1 chimera”).

### Aptamer-siRNA Chimera Specifically Silences Gene Expression

Using similar methods as described in Example 2, the aptamer-siRNA chimera, A9-Plk1 chimera, was assayed for its ability to deliver siRNAs against Plk1 to PC-3 cells or LNCaP cells in cell culture. In these cell cultures, cells were treated by contacting the cells with either transfection reagent alone (FIG. 9, lane 1), 400 nM Plk1 siRNA with transfection reagent (FIG. 9, lane 2), and various concentrations of A9-Plk1 chimera (in the absence of transfection reagent) on once a day for three consecutive days (FIG. 9, lane 3, 300 nM; FIG. 9, lane 4, 150 nM; and FIG. 9, lane 5, 75 nm). Cells were collected from the cultures on day 4 for analysis. Gene silencing was assessed by immunoblotting using antibodies specific to human PLK1.

As shown in FIG. 9, the level of Plk1 silencing in LNCaP cells achieved with all concentrations of A9-Plk1 chimera tested (lanes 3-5) is comparable to that achieved with using Plk1 siRNA in transfection reagent (lane 2). Thus, these results show that a nucleic acid molecule comprising a targeting moiety (an aptamer) and a dsRNA molecule (siRNA), wherein the targeting moiety and at least one strand of the dsRNA molecule are a contiguous nucleic acid molecule, can be contacted with the target cells, and internalized into targeted cells. Inside the targeted cells, the siRNA is delivered to the siRNA processing machinery in the cytoplasm (e.g., Dicer) such that gene silencing is effected.

### EXAMPLE 4

This is another illustrative example describing and demonstrating the invention. Provided is a method of targeted delivery of an RNA silencing moiety, the method comprising contacting a nucleic acid molecule and cells in conditions effective for the nucleic acid molecule to deliver the RNA silencing moiety into the cell cytoplasm such that the RNA silencing moiety is bound and processed by Dicer (i.e., is a Dicer substrate). This method involves the use of a nucleic acid molecule composed of (i) a RNA silencing moiety, comprised of dsRNA comprising a guide strand to be delivered to Dicer; and (ii) and a targeting moiety for binding a cell and resultant cellular internalization to deliver the dsRNA to the cell cytoplasm to be accessible by Dicer, wherein the targeting moiety is an aptamer; and wherein the targeting moiety and at least one strand of the RNA silencing moiety are a contiguous nucleic acid molecule. Also provided is a method for making a Dicer substrate, comprising: (a) synthesizing a nucleic acid molecule comprising (i) a targeting moiety comprising an aptamer, and (ii) a first single stranded RNA (e.g., an RNA molecule comprising either a guide strand or a passenger strand) for forming a dsRNA; wherein the targeting moiety and the first single stranded RNA are a contiguous nucleic acid molecule; (b) hybridizing a second single stranded RNA, having full complementarity or partial complementarity to the first single stranded RNA, to the first single stranded RNA of the synthesized RNA molecule in forming a dsRNA which can act as a Dicer substrate; and wherein the targeting moiety is capable of binding to a receptor on the surface of a cell and subsequently internalizing the nucleic acid molecule so that the dsRNA is accessible to Dicer in the cell cytoplasm. Also provided is a method of introducing an RNA silencing moiety comprised of dsRNA into cells, the method comprising contacting cells with a nucleic acid molecule under conditions effective for a targeting moiety of the nucleic acid molecule to bind to a receptor on the cells which, upon binding, then results in internalization of the nucleic acid molecule into the cells; wherein the nucleic acid molecule comprises a targeting moiety and a dsRNA molecule, wherein the targeting moiety and at least one strand of the dsRNA molecule are a contiguous nucleic acid molecule, wherein the targeting molecule is an aptamer that recognizes a cell surface receptor on the cells, and wherein a guide strand of the dsRNA becomes bound to Dicer subsequent to introduction of the nucleic acid molecule into the cells.

In this example, the following sequences were used to illustrate the invention.

Plk1 siRNA target sequence:
(SEQ ID NO: 2)
AAGGGCGGCTTTGCCAAGTGC
9.38-Plk1 Sense Strand (the latter, in bold type):
(SEQ ID NO: 21)
5′GGGAGAGAGGAAGAGGGATGGGCTCTACGTCGATCGAATTTCGGAT
GCATCCCAAATACCAGCATAACCCAGAGGTCGATAGTACTGGATCCCA
AGGGCGGCUUUGCCAAGUGC3′
Plk1 Antisense siRNA:
(SEQ ID NO: 9)
5′GCACUUGGCAAAGCCGCCCdTdT3′

### Nucleic Acid Molecules

A nucleic acid molecule comprising a targeting moiety (in this Example, aptamer 9.38) and a dsRNA molecule (in this Example, targeting Plk1), wherein the targeting moiety and at least one strand of the dsRNA molecule are a contiguous nucleic acid molecule, were generated in order to specifically target siRNAs to cells expressing the cell-surface receptor c-Kit (Stem Cell Factor Receptor). The aptamer portion of the chimera (9.38) mediates binding to c-Kit and subsequent internalization. The siRNA portion targets the expression of survival gene, Plk1. Thus, one representative nucleic acid molecule comprising a targeting moiety and an RNA silencing moiety is illustrated by a molecule in which the nucleic acid sequence of SEQ ID NO:21 is hybridized to a nucleic acid sequence of SEQ ID NO:9 (in forming “9.38-Plk1 chimera”).

Binding of Aptamer-siRNA Chimera to c-Kit

The binding of aptamer 9.38-Plk1 chimera (with lower strand annealed) was investigated by double-filter nitrocellulose filter binding assays. All binding studies were performed in physiologic binding buffer (20 mM HEPES, pH 7.4, 150 mM NaCl, 2 mM CaCl₂, and 0.01% BSA) at 37° C. The results of these assays are shown in FIG. 10.

### Aptamer-siRNA Chimera Specifically Silences Gene Expression

In making the aptamer-RNA-silencing moiety chimera, in vitro transcribed aptamer and a first strand of the siRNA were annealed to a second strand of RNA having full complementary to the first strand of RNA at a 1:1 ratio. To investigate the ability of aptamer 9.38 to deliver siRNAs to cells, the 9.38-Plk1 chimera was added to DLD-1 cells (12 well plates at 3 million cells per well) at 800 nM, and again after two days at 400 nm concentration. On day 5, cells were harvested, centrifuged, and lysed. Debris was removed by centrifugation, and samples of supernatant were loaded on a polyacrylamide gel. Proteins were separated, and transferred to membrane and analyzed by Western blot analysis with a PLK1-specific antibody. PLK1 protein expression was considerably lower in the sample treated with the 9.38-Plk1 chimera compared to the assay negative controls (mutant 9.38-Plk1 chimera, and 9.38-CON chimera). The experiment was repeated with K562 cells, which lack detectable c-Kit on their cell surface. No knockdown of Plk1 expression was detected in K562 cells. These results suggest that the 9.38-Plk1 chimera was delivered specifically into cells expressing a cell surface receptor of c-Kit, internalized such that the RNAZ silencing moiety can be delivered to the cytoplasm to be accessed by Dicer, and resulted in gene knockdown.

### EXAMPLE 5

This is another illustrative example describing and demonstrating the invention. Provided is a method of targeted delivery (to a specific cell type or cell population) of an RNA silencing moiety, the method comprising contacting a nucleic acid molecule and cells in conditions effective for the nucleic acid molecule to deliver the RNA silencing moiety into the cell cytoplasm such that the RNA silencing moiety is bound and processed by Dicer (i.e., is a Dicer substrate). This method involves the use of a nucleic acid molecule comprised of (i) a RNA silencing moiety, comprised of dsRNA comprising a guide strand to be delivered to Dicer; and (ii) and a targeting moiety (in this Example, a DNA molecule) for binding a receptor on a cell and resultant cellular internalization of the nucleic acid molecule to deliver the dsRNA to the cell cytoplasm to be accessible by Dicer, wherein the targeting moiety is an aptamer; and wherein the targeting moiety and at least one strand of the RNA silencing moiety are a contiguous nucleic acid molecule. Also provided is a method for making a Dicer substrate, comprising: (a) synthesizing a nucleic acid molecule comprising (i) a targeting moiety comprising a DNA aptamer, and (ii) a first single stranded RNA (e.g., an RNA molecule comprising either a guide strand or a passenger strand) for forming a dsRNA; wherein the targeting moiety and the first single stranded RNA are a contiguous nucleic acid molecule; (b) hybridizing a second single stranded RNA, having full complementarity or partial complementarity to the first single stranded RNA, to the first single stranded RNA of the synthesized RNA molecule in forming a dsRNA which can act as a Dicer substrate; and wherein the targeting moiety is capable of binding to a receptor on the surface of a cell and subsequently being internalizing into the cell in delivering the dsRNA into the cell cytoplasm for access by Dicer. Also provided is a method of introducing an RNA silencing moiety comprised of dsRNA into cells, the method comprising contacting cells with a nucleic acid molecule under conditions by which a targeting moiety of the nucleic acid molecule binds to a receptor on the cells, resulting in internalization of the nucleic acid molecule into the cells; wherein the nucleic acid molecule comprises a targeting moiety and a dsRNA molecule, wherein the targeting moiety and at least one strand of the dsRNA molecule are a contiguous nucleic acid molecule, wherein the targeting molecule is an aptamer that recognizes a cell surface receptor on the cells, and wherein a guide strand of the dsRNA becomes bound to Dicer subsequent to introduction of the nucleic acid molecule into the cells. In this example, illustrated is an aptamer that mediates transport of the RNA silencing moiety into the nucleus of a cell. The RNA silencing moiety is processed by Drosha in the nucleus prior to being transported to the cytoplasm of the cell for becoming a Dicer substrate.

In this example; the following sequences were used to illustrate the invention.

Plk1 siRNA target sequence:
(SEQ ID NO: 2)
AAGGGCGGCTTTGCCAAGTGC
AS1411-Plk1 Sense Strand (the latter, in bold
type):
(SEQ ID NO: 22)
5′TGGTGGTGGTGGTTGTGGTGGTGGTGGAAGGGCGGCUUUGCCAAG
UGC3′
Complementary RNA
(SEQ ID NO: 23)
UUCCCGCCGAAACGGUUCACGAA

### Nucleic Acid Molecules

A nucleic acid molecule comprising a targeting moiety (in this Example, DNA aptamer AS1411) and a dsRNA molecule (in this Example, targeting Plk1), wherein the targeting moiety and at least one strand of the dsRNA molecule are a contiguous nucleic acid molecule, were generated in order to specifically target siRNAs to cells expressing the cell-surface receptor nucleolin. The aptamer portion of the chimera (AS1411) mediates binding to nucleolin and subsequent internalization. The siRNA portion targets the expression of survival gene, Plk1. Thus, one representative nucleic acid molecule comprising a targeting moiety and an RNA silencing moiety is illustrated by a molecule in which the nucleic acid sequence of SEQ ID NO:22 is hybridized to a nucleic acid sequence of SEQ ID NO:23 (in forming “AS1411-Plk1 chimera”).

### Binding and Internalization of AS1411 Aptamer

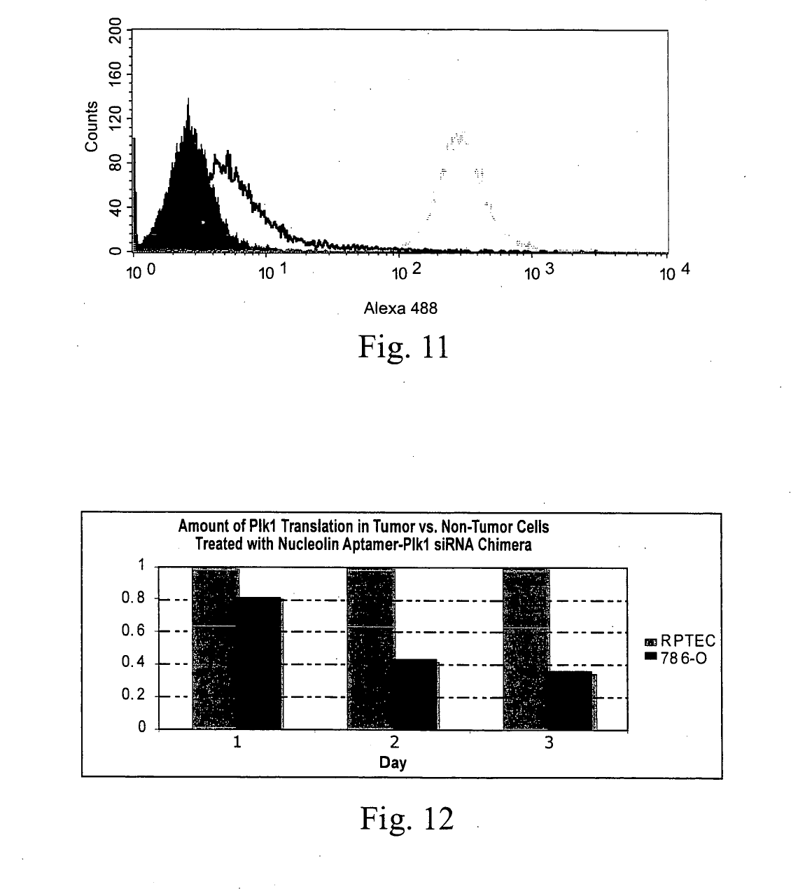

Using methods similar to those described in Example 2 herein, binding and internalization was assayed by flow cytometry. Briefly, HeLa cells (expressing nucleolin as a cell surface receptor) were incubated with AS1411 aptamer or an assay negative control aptamer (AS1411 aptamer with an RNA sequence lacking detectable binding to HeLa cells; “Control Aptamer”) that were fluorescently labeled (Alexa 488), and at a concentration of 1 μM overnight in DMEM 10% FBS. The next day, cells were trypsinized and washed three times with PBS lacking magnesium and calcium (the structure of aptamer AS1411 requires magnesium, so washing with PBS lacking magnesium should denature an wash away non-internalized aptamer). The cells were then analyzed by flow cytometry. In referring to FIG. 11, the x-axis shows the intensity of the fluorescence, and the y-axis shows the amount of cells at that intensity. In FIG. 11, the shaded peak represents fluorescence of unstained HeLa cells, the peak outlined with heavy lining represents fluorescence of HeLa cells incubated with Control Aptamer, and the peak outlined with light lining represents fluorescence of HeLa cells incubated with AS1411 aptamer. These results show that a targeting moiety comprising a DNA aptamer can be contacted with cells expressing a cell surface receptor for which the targeting moiety has binding specificity (“target cells”), with subsequent internalization of the targeting moiety into the target cells.

### Transport to Nucleus of Cells, Once Internalized

HeLa cells were cultured in multi-well plates with glass cover slides on Day 0. On Day 1, fluorescent (Alexa 488)-labeled AS1411 aptamer was incubated with the HeLa cells at a concentration of 400 nM in culture medium (Opti-MEM) at 37° C., 5% CO₂ for either 2 minutes, 10 minutes, or 45 minutes. After the indicated amount of time, the cells were then washed, fixed in 4% paraformaldehyde, counter-stained with DAPI and analyzed by fluorescent microscopy. Analysis by fluorescent microscopy showed: at 2 minutes, some of the AS1411 aptamer is detected as bound to the cell membrane, and some is also detected in the cytoplasm; at 10 minutes, some of the AS1411 aptamer is in the cytoplasm and some of it has also entered the nuclei; and at 45 minutes, the aptamer is exclusively detected in the cell nuclei. Similar results were obtained using cells, other than HeLa cells, expressing nucleolin as a cell surface receptor; including, but not limited to, tumor cell lines 786-O (renal tumor) cells, HCC-1806 (breast tumor) cells, and Mia (pancreatic tumor) cells. These results show that a targeting moiety comprising a DNA aptamer can be contacted with cells expressing a cell surface receptor for which the targeting moiety has binding specificity, with subsequent internalization of the targeting moiety into the target cells in one or more of the cytoplasm and nuclei of the target cells.

This study was repeated, but instead the AS1411-Plk1 chimera was analyzed for internalization into HeLa cells. The AS1411-Plk1 chimera was labeled with fluorescein isothiocyanate (FITC) and then contacted (at a concentration of 400 nM) with HeLa cells at 37° C., 5% CO₂ for 1 hour. After the 1 hour incubation, the cells were then washed, fixed in 4% paraformaldehyde, counter-stained with DAPI, and analyzed by fluorescent microscopy. Analysis by fluorescent microscopy showed that all of the labeled AS1411-Plk1 chimera had localized into the nuclei of the cells. These results show that a nucleic acid molecule comprised of a targeting moiety comprising a DNA aptamer and an RNA silencing moiety (dsRNA) can be contacted with cells expressing a cell surface receptor for which the targeting moiety has binding specificity, with subsequent internalization of the nucleic acid molecule into the target cells and localization of the nucleic acid molecule into the nuclei of the target cells.

### Aptamer-siRNA Chimera Specifically Silences Gene Expression

Using similar methods as described in Example 2, the aptamer-siRNA chimera, AS1411-Plk1 chimera, was assayed for its ability to deliver siRNAs against Plk1 to renal proximal tubule epithelial (noncancerous) cells or 786-O cells (cancer cell line) in cell culture. In these cell cultures, cells were treated for each of 3 days by contacting the cells with AS1411-Plk1 chimera (400 nM each day, in the absence of transfection reagent). Each day the cells were also pulsed with media containing S35-radioisotope, so that the protein translated on that day would be labeled by S35. The cells were then lysed and PLK1 and a control protein (beta tubulin) are immunoprecipitated, run on a gel, and quantified by radiography and scanning. As shown in FIG. 12, the level of PLK1 dropped in the tumor cells, as compared to the non-cancerous cells, showing that the AS1411-Plk1 chimera selectively internalized into and delivered the RNA silencing moiety to the tumor cells (e.g., to the RNA silencing processing machinery, such as Dicer, and then to RISC to effect silencing). Thus, these results show that a nucleic acid molecule comprising a targeting moiety (an aptamer) and a dsRNA molecule (siRNA), wherein the targeting moiety and at least one strand of the dsRNA molecule are a contiguous nucleic acid molecule, can be contacted with the target cells, and internalized into targeted cells. Inside the targeted cells, the siRNA is delivered to the siRNA processing machinery in the cytoplasm (e.g., Dicer) such that gene silencing is effected.

### EXAMPLE 6

In this example of illustrating the methods and compositions of the present invention, used is a novel class of RNA silencing moiety termed a microRNA-based siRNA or “misiRNA”. In describing misiRNA, keep in mind the following. The siRNA portion of the misiRNA generally has perfect or near-perfect Watson-Crick base pairing, both in the double-stranded precursors and in the pairing of the guide strand with the target mRNA. “Near perfect” is defined as no less than 1 and no more than 3 mismatched (non-base paired) bases between the guide strand and the target mRNA that it binds. The misiRNA is processed in the nucleus by Drosha (RNAse III family enzyme). misiRNA share major common features, as follows.

An misiRNA:
(a) comprises an siRNA portion that has full complementarity (perfect) or near-perfect Watson-Crick base pairing (e.g., only one or two non-base paired nucleotides), both in the double-stranded precursors and in the pairing of the guide strand with the target mRNA;
(b) comprises a secondary structure, a hairpin comprising a stem-loop structure, that can easily be detected by using commercially available computer software that predicts folding into secondary structure (e.g., RNAfold program; see also, microPred; Batuwita and Palade, Bioinformatics, 2009 Apr. 15; 25(8):989-95);
(c) comprises a length of about 50 nucleotides to about 300 nucleotides, and alternatively from about 75 nucleotides to about 150 nucleotides long, and alternatively from about 100 nucleotides to about 125 nucleotides long;
(d) comprises a stem region of about 30% to 90% of the length of the misiRNa (e.g., for an misiRNA of a length of about 100 nucleotides, the stem region comprises about 30 to 90 nucleotides), and alternatively of about 50% to 75% of the length of the misiRNa, and the stem region may comprise a perfectly base-paired duplex or may further comprise along the stem region one or more “bulges” (e.g., of 1 to 4 nucleotides that are not base-paired with any other nucleotide in the stem region, thereby causing a bulging structure in the stem region), preferably the number of bulges is between 0 and not more than 5, and alternatively between 1 and 3;
(e) comprises a terminal loop portion of greater than 5 nucleotides in size, and alternatively more than 10 nucleotides but less than 25 nucleotides in size;
(f) comprises a terminus, located on the opposite end of misiRNA from the terminal loop portion, that has zero single stranded nucleotide overhangs, or may comprise at least one single stranded nucleotide overhang, wherein when present each single stranded nucleotide overhang is six or fewer nucleotides in length, or alternatively between 1 to nucleotides in length;
(g) comprises modified RNA bases, the modification promoting stability of the misiRNA or the siRNA portion of the misiRNA to RNA endonucleases other than Drosha or Dicer (e.g, a 2′-sugar modification of either purines or pyrimidines, or both purines and pyrimidines); and
(h) comprises a Drosha substrate, and is thereby recognized and cleaved by Drosha to a smaller size (e.g., about 30 nucleotides to about 70 nucleotides) molecule which is transported to the cytoplasm for cleavage by Dicer to an siRNA.
   Also, because misiRNA comprise a length of about 50 nucleotides to about 300 nucleotides, misiRNA may comprise an RNA silencing moiety that comprises more than one guide strand (e.g., each guide strand targeting the same mRNA or a different mRNA) for RNA silencing. For example, a first guide strand and a second guide strand may be on the same RNA strand (synthesized is a contiguous RNA strand containing a nucleotide sequence that is composed of both the first guide strand and the second guide RNA) or be on opposite RNA strands of the RNA silencing moiety, such that upon Drosha processing, produced is more than one dsRNA that then may be transported to the cytoplasm to act as Dicer substrates. Nucleotides that are the signal for Drosha processing (as known to those skilled in the art) can be spaced apart between the two or more guide strands to direct Drosha to cleave the misiRNA to produce two or more dsRNAs that are then transported into the cytoplasm to be accessible to Dicer.

misiRNA are novel in that not described are: (a) a precursor to siRNA that is processed by Drosha with the resultant Drosha-processed molecule being subsequently transported to the cytoplasm and then processed by Dicer as siRNA; (b) processing of an siRNA precursor in the nuclei of cells; and (c) processing of an RNA silencing moiety with 2′-sugar modifications by Drosha. Additionally, it is the current view by those skilled in the art that siRNA need be restricted to less than 30 bp in almost all mammalian cells (with one exception reported for mouse oocytes), because introduction of siRNA of 38 bp or greater have shown toxic effect to mammalian cells, exhibited by one or more of interferon induction, nonspecific inhibition of protein synthesis, RNA degradation, and nonspecific gene expression (see, e.g., Chang et al., 2009, Mol. Cells, 27(6):689-695; Stein et al., Dev Biol. 2005 Oct. 15; 286 (2): 464-71; Paddison et al., Methods Mol Biol. 2004; 265: 85-100).

misiRNA can be synthesized by any method known to those skilled in the art for nucleic acid synthesis, including but not limited to, chemical synthesis (e.g., linear synthesis, fragment synthesis (synthesis of portions) followed by assembly of the fragments to the desired complete nucleic acid molecule, or a combination thereof), enzymatic synthesis (e.g., primer extension or transcription using a polymerase), cleavage from a larger precursor, recombinant synthesis, or a combination thereof. misiRNA can be introduced into mammalian cells using any method or delivery vehicle known to those skilled in the art, including but not limited to via a transfection reagent, a lipid-based or polymer-based microparticle or nanoparticle or a lipid and polymer-based microparticle or nanoparticle, or by a targeting moiety comprising an aptamer as described herein, or a combination thereof, and using methods known to those skilled in the art.

In this example, the following sequences were used to illustrate the invention.

Plk1 siRNA target sequence:
(SEQ ID NO: 2)
AAGGGCGGCTTTGCCAAGTGC
AS1411-misiRNAPlk1 Sense Strand (the latter, in
bold type):
(SEQ ID NO: 24)
TGGTGGTGGTGGTTGTGGTGGTGGTGGGCCUGGCCUCCUGCAGUGCCA
CGCUAUGCACUUGGGAAAGCCGCCCUAGUUGGACACUCCAUGUGGUAG
AGUAAGGGCGGCUUUGCCAAGUGCUUAGUGCGGCACAUGCUUACCAGC
UCUCUAGGC
Complementary RNA:
(SEQ ID NO: 25)
CACAUGCUUACCAGCUCUAGGC
AS1411-CON:
(SEQ ID NO: 26)
TGGTGGTGGTGGTTGTGGTGGTGGTGGUCAAGAAGCCAAGGAUAAU

### Nucleic Acid Molecules

A nucleic acid molecule comprising a targeting moiety (in this Example, DNA aptamer AS1411) and a dsRNA molecule (in this Example, comprising a misiRNA targeting Plk1), wherein the targeting moiety and at least one strand of the dsRNA molecule are a contiguous nucleic acid molecule, were generated in order to specifically target siRNAs to cells expressing the cell-surface receptor nucleolin. The aptamer portion of the chimera (AS1411) mediates binding to nucleolin and subsequent internalization. The misiRNA portion targets the expression of survival gene, Plk1. For example, the nucleolin aptamer with the extension for the misiRNA (e.g., SEQ ID NO:24) was mixed in PBS with 50 mM MgCl₂, heated to 98° C. for two minutes, and then cooled to 37° C. for 5 minutes. The misiRNA complementary RNA to be annealed (e.g., SEQ ID NO:25) was then added at two-fold molar ratio. The mixture was then heated to 55° C. for 5 minutes, and then cooled to 37° C. for 5 minutes. The chimeras were then added to cell growth media.

### Internalization, Followed by Transport to Nucleus of Cells

Similar to the methods described above for the AS1411-Plk1 chimera, the AS1411-misiRNAPlk1 chimera was analyzed for internalization into HeLa cells. The AS1411-misiRNAPlk1 chimera was labeled with fluorescein isothiocyanate (FITC) and then contacted (at a concentration of 400 nM) with HeLa cells at 37° C., 5% CO₂ for 1 hour. After the 1 hour incubation, the cells were then washed, fixed in 4% paraformaldehyde, counter-stained with DAPI, and analyzed by fluorescent microscopy. Analysis by fluorescent microscopy showed that all of the labeled AS1411-misiRNAPlk1 chimera had localized into the nuclei of the cells. These results show that a nucleic acid molecule comprised of a targeting moiety comprising a DNA aptamer and an RNA silencing moiety (dsRNA) comprising an misiRNA can be contacted with cells expressing a cell surface receptor for which the targeting moiety has binding specificity, with subsequent internalization of the nucleic acid molecule into the target cells and localization of the nucleic acid molecule into the nuclei of the target cells.

### Aptamer-misiRNA Chimera Specifically Silences Gene Expression

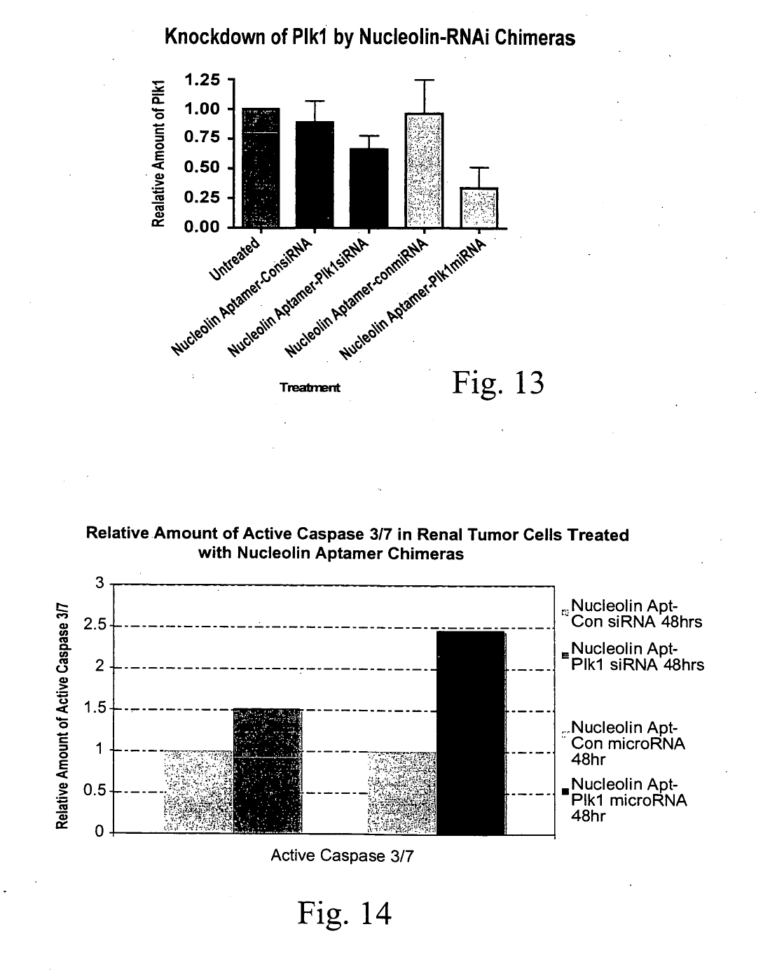

Using the methods for assessing Plk1 knockdown in Example 5 for the AS1411-siRNA chimera, the experiment was repeated to compare the level of knockdown of Plk1 achieved with the AS1411-siRNA chimera versus the level of knockdown of Plk1 achieved with the AS1411-misiRNA chimera. As shown in FIG. 13, surprisingly, the AS1411-misiRNA chimera (“Nucleolin Aptamer-Plk1miRNA”) was significantly more effective in silencing gene expression than the AS1411-siRNA chimera (“Nucleolin Aptamer-Plk1siRNA”)

### Knockdown of Gene Expression by Aptamer-siRNA Chimera or Aptamer-misiRNA Chimera Results in Functional Changes of Cells Expressing Surface Receptor Nucleolin

As another indication of gene silencing mediated by a method according to the present invention, assayed was the ability of an aptamer-siRNA chimera or the aptamer-misiRNA chimera (wherein the RNA silencing moiety targets an oncogene or anti-apoptotic genes) to induce apoptosis. The amount of apoptosis in a population of cells was measured by quantifying the amount of active caspases 3 and 7 (“3/7”) present on a Western blot (using antibodies specific for the respective human caspases). 786-O primary renal adenocarcinoma cells were treated with either AS1411-Plk1 chimera or AS1411-Plk1misiRNA chimera or an AS1411-CON chimera (assay controls, for 48 hours. Knocking down expression of Plk1 in cells results in a reduction of anti-apoptotic protein PLK1 produced, and therefore, would result in an increase in apoptosis, as measured by an increase in the levels of active caspase 3 and 7. As shown in FIG. 14, cells treated with either AS1411-Plk1 chimera (“Nucleolin Apt-Plk1 siRNA”) or AS1411-Plk1misiRNA chimera (“Nucleolin Apt-Plk1 microRNA”) show a significant increase in caspase 3 and 7 activity, relative to the respective assay negative control chimeras (“Nucleolin Apt-Con siRNA”, and “Nucleolin Apt-Con microRNA”), indicating successful knockdown of Plk1 gene expression. As an unexpected result, and as also shown in FIG. 14, the aptamer-misiRNA chimera produced a significant and more robust induction of caspases than did the aptamer-siRNA chimera, indicating that the aptamer-misiRNA chimera was more effective than an aptamer-siRNA chimera in mediating RNA silencing (as represented in this Example by knockdown of Plk1 gene expression). Thus, misiRNA are molecules that appear superior to siRNA in RNA silencing applications.

All documents and other information sources cited above are hereby incorporated in their entirety by reference.

1. A method of targeted delivery of an RNA silencing moiety for RNA silencing, the method comprising contacting a nucleic acid molecule with cells in conditions effective for the nucleic acid molecule to deliver the RNA silencing moiety into the cells such that the RNA silencing moiety is capable of being bound and processed by Dicer, wherein:
(a) the nucleic acid molecule comprises (i) a targeting moiety comprising an aptamer, and (ii) a first single stranded RNA, comprising either a guide strand or a passenger strand for forming a dsRNA, wherein the targeting moiety and the first single stranded RNA are a contiguous nucleic acid molecule;
(b) hybridized to the first single stranded RNA is a second single stranded RNA having full complementarity or partial complementarity to the first single stranded RNA, in forming an RNA silencing moiety comprising a dsRNA which can act as a Dicer substrate; and
(c) the aptamer is capable of binding a surface receptor on the cells to target delivery to the cells which, upon binding, results in internalization of the nucleic acid molecule into the cells in delivering the RNA silencing moiety to be accessible to Dicer.

2.-10. (canceled)

11. A method of RNA silencing comprising contacting cells with a nucleic acid molecule under conditions by which a targeting moiety of the nucleic acid molecule, when contacted with the cells, binds to a surface receptor on the cells and results in internalization of the nucleic acid molecule into the cells; wherein the nucleic acid molecule comprises a targeting moiety portion that is an aptamer, and a RNA silencing moiety portion that is a dsRNA, wherein the aptamer and at least one strand of the dsRNA are a contiguous nucleic acid molecule; and wherein a guide strand of the dsRNA becomes bound to Dicer subsequent to introduction of the nucleic acid molecule into the cells, and is processed in the cells to result in RNA silencing of a gene that is targeted in the cells.

12.-16. (canceled)

17. A chimeric nucleic acid molecule comprising a targeting moiety portion and an RNA silencing moiety portion, wherein:
(a) the targeting moiety portion is an aptamer, and the aptamer and at least one strand of the RNA silencing moiety portion form a contiguous nucleic acid molecule composed of nucleotides;
(b) the RNA silencing moiety portion is dsRNA composed of two strands having full complementarity or partial complementarity with respect to each other;
(c) the aptamer is capable of binding to a surface receptor on a cell which, upon binding, results in internalization of the chimeric nucleic acid molecule into the cell and transport of the RNA silencing moiety portion to become accessible to Dicer;
(d) the RNA silencing moiety portion comprises a Dicer substrate; and
(e) at least some of the nucleotides of the chimeric nucleic acid molecule have a 2′-sugar modification.

18.-23. (canceled)

24. A compound comprising:
a targeting moiety, which specifically binds to a disease related cell surface marker,
a nucleic acid moiety which specifically induces cell death and
a linker, which covalently links the targeting moiety to the nucleic acid moiety.

25. The compound of claim 24, wherein the linker is a disulfide bond, a phosphodiester bond, a phosphothioate bond, an amide bond, an amine bond, a thioether bond, an ether bond, an ester bond or a carbon-carbon bond.

26. The compound of claim 24, wherein the targeting moiety is a nucleic acid or a polypeptide.

27. The compound of claim 24, wherein the targeting moiety is a binding ligand for a cell surface receptor.

28. The compound of claim 24, wherein the targeting moiety is at least one aptamer, an antibody, a diabody or a derivative or fragment of an antibody.

29. The compound of claim 28, wherein the targeting moiety is represented by at least two aptamers.

30. The compound of claim 24, wherein the targeting moiety is selected from the group consisting of carbohydrates, lipids, vitamins, small receptor ligands, nucleic acids, cell surface carbohydrate binding proteins and their ligands, lectins, r-type lectins, galectins, ligands to the cluster of differentiation (CD) antigens, CD30, CD40, cytokines, chemokines, colony stimulating factors, type-1 cytokines, type-2 cytokines, interferons, interleukins, lymphokines, monokines, mutants, derivatives and/or combinations of any of the above.

31. The compound of claim 24, wherein the disease related cell surface marker is selected from the group consisting of CD antigens, cytokine receptors, hormone receptors, growth factor receptors, ion pumps, channel-forming proteins, multimeric extracellular matrix proteins, metallo proteases, Her3 or PSMA.

32. The compound of claim 24, wherein the targeting moiety binds to a cell surface receptor of a target cell and mediates subsequent translocation of the compound into the cytosol of the target cell.

33. The compound of claim 32, wherein after translocation of the compound into the target cell the nucleic acid moiety induces cell death of the target cell.

34. The compound of claim 24, wherein the nucleic acid moiety is a siRNA, a shRNA an antisense DNA or RNA, a dsRNA or a miRNA.

35. The compound of claim 24, wherein the nucleic acid moiety comprises 10 to 40 nucleic acid base pairs or nucleic acid bases.

36. The compound of claim 24, wherein the nucleic acid moiety is specifically inhibitory to activity of eukaryotic elongation factor 2 (eEF-2), homologues of eEF-2 or analogues of eEF-2.

37. The compound of claim 24, wherein the nucleic acid moiety is specifically inhibitory to activity of apoptosis inhibitors Bcl2, Bcl-XL, Bcl-W, Mcl-1, A1, Ced9, E1B19K, BHRF1, Bag-1, Raf-1, Calcineurin, Smn, Beclin, ANT and VDAC, IAP-1, IAP-2, Survivin, x-IAP, IKK-α, IκB, NF-κB, FLIP, PI3K or PDK1.

38. The compound of claim 24 comprising an aptamer and the nucleic acid moiety linked by a phosphodiester or by a phosphothioate bond.

39. The compound of claim 24 comprising an antibody and a RNA linked by a disulfide bond.

40. The compound of claim 38 consisting of an RNA.

41. The compound of claim 24, wherein the nucleic acid moiety does not induce cell death and down-regulates a specific key element of a regulatory pathway of the target cell.

42. A DNA coding for the RNA of claim 40.

43. A cell, an organ or a non-human animal transfected with a RNA or DNA encoding a compound comprising:
a targeting moiety, which specifically binds to a disease related cell surface marker,
a nucleic acid moiety which specifically induces cell death and
a linker, which covalently links the targeting moiety to the nucleic acid moiety.

44. The compound of claim 24 further comprising a moiety, which enables purification and/or detection of the compound, facilitates translocation of the compound into the target cell and/or intracellular separation therein, and/or activates the nucleic acid.

45. A method of treating a condition comprising preparing a medicament comprising:
a targeting moiety which specifically binds to a disease related cell surface marker,
a nucleic acid moiety which specifically induces cell death and
a linker, which covalently links the targeting moiety to the nucleic acid moiety.

46. The method of claim 45, wherein the medicament is administered locally or systemically or in combination with other therapeutic efficacy enhancing compounds.

47. The method of claim 45 further comprising treating a patient for the condition, with the condition being a cancerous proliferative disease, a non-cancerous proliferative disease, allergy, autoimmune disease, chronic inflammation, or infections.

48. The compound of claim 24, wherein the linker is a phosphodiester bond.

49. The compound of claim 24, wherein the targeting moiety is a nucleic acid.

50. The compound of claim 24, wherein the targeting moiety is at least one aptamer.

51. The compound of claim 24, wherein the disease related cell surface marker is selected from the group consisting of CD antigens, hormone receptors and PSMA.

52. The compound of claim 24, wherein the nucleic acid moiety is a siRNA, a shRNA or a miRNA.

53. The compound of claim 24, wherein the nucleic acid moiety is specifically inhibitory to activity of Bcl2.

54. The compound of claim 24 comprising an aptamer and the nucleic acid moiety linked by a phosphodiester bond.

55. The method of claim 45 further comprising treating a patient for the condition, with the condition being a cancerous proliferative disease or an infection.


SEQUENCE LISTING (26 sequences)

SEQ ID NO: 1 - DNA, 21 bp, Artificial Sequence
  Description of Artificial Sequence Synthetic oligonucleotide
aattctccgaacgtgtcacgt

SEQ ID NO: 2 - DNA, 21 bp, Artificial Sequence
  Description of Artificial Sequence Synthetic oligonucleotide
aagggcggctttgccaagtgc

SEQ ID NO: 3 - DNA, 21 bp, Artificial Sequence
  Description of Artificial Sequence Synthetic oligonucleotide
nngtgaagtcaacatgcctgc

SEQ ID NO: 4 - DNA, 21 bp, Artificial Sequence
  Description of Artificial Sequence Synthetic oligonucleotide
nncctcaccaatgggtccttt

SEQ ID NO: 5 - RNA, 71 nt, Artificial Sequence
  Description of Artificial Sequence Synthetic oligonucleotide
gggaggacgaugcggaucagccauguuuacgucacuccuugucaauccucaucggcagacgacucgcccga

SEQ ID NO: 6 - RNA, 92 nt, Artificial Sequence
  Description of Artificial Sequence Synthetic oligonucleotide
gggaggacgaugcggaucagccauguuuacgucacuccuugucaauccucaucggcagacgacucgcccgaaauucuccgaacgugucacgu

SEQ ID NO: 7 - DNA, 21 bp, Artificial Sequence
  Description of Artificial Sequence Synthetic oligonucleotide
acgugacacguucggagaatt

SEQ ID NO: 8 - RNA, 92 nt, Artificial Sequence
  Description of Artificial Sequence Synthetic oligonucleotide
gggaggacgaugcggaucagccauguuuacgucacuccuugucaauccucaucggcagacgacucgcccgaaagggcggcuuugccaagugc

SEQ ID NO: 9 - DNA, 21 bp, Artificial Sequence
  Description of Artificial Sequence Synthetic oligonucleotide
gcacuuggcaaagccgccctt

SEQ ID NO: 10 - RNA, 92 nt, Artificial Sequence
  Description of Artificial Sequence Synthetic oligonucleotide
gggaggacgaugcggaucagccauguuuacgucacuccuugucaauccucaucggcagacgacucgcccgaaagugaagucaacaugccugc

SEQ ID NO: 11 - RNA, 21 nt, Artificial Sequence
  Description of Artificial Sequence Synthetic oligonucleotide
gcaggcauguugacuucacuu

SEQ ID NO: 12 - RNA, 92 nt, Artificial Sequence
  Description of Artificial Sequence Synthetic oligonucleotide
gggaggacgaugcggaucagccauccuuacgucacuccuugucaauccucaucggcagacgacucgcccgaaagggcggcuuugccaagugc

SEQ ID NO: 13 - DNA, 32 bp, Artificial Sequence
  Description of Artificial Sequence Synthetic primer
taatacgactcactatagggaggacgatgcgg

SEQ ID NO: 14 - DNA, 16 bp, Artificial Sequence
  Description of Artificial Sequence Synthetic primer
tcgggcgagtcgtctg

SEQ ID NO: 15 - DNA, 71 bp, Artificial Sequence
  Description of Artificial Sequence Synthetic primer
gggaggacgatgcggatcagccatgtttacgtcactccttgtcaatcctcatcggcagacgactcgcccga

SEQ ID NO: 16 - DNA, 37 bp, Artificial Sequence
  Description of Artificial Sequence Synthetic primer
acgtgacacgttcggagaatttcgggcgagtcgtctg

SEQ ID NO: 17 - DNA, 37 bp, Artificial Sequence
  Description of Artificial Sequence Synthetic primer
gcacttggcaaagccgccctttcgggcgagtcgtctg

SEQ ID NO: 18 - DNA, 37 bp, Artificial Sequence
  Description of Artificial Sequence Synthetic primer
gcaggcatgttgacttcactttcgggcgagtcgtctg

SEQ ID NO: 19 - DNA, 31 bp, Artificial Sequence
  Description of Artificial Sequence Synthetic primer
aggacgatgcggatcagccatccttacgtca

SEQ ID NO: 20 - RNA, 91 nt, Artificial Sequence
  Description of Artificial Sequence Synthetic oligonucleotide
gggaggacgaugcggaccgaaaaagaccugacuucuauacuaagucuacguucccagacgacucgcccgaaagggcggcuuugccaagugc

SEQ ID NO: 21 - DNA, 114 bp, Artificial Sequence
  Description of Artificial Sequence Synthetic polynucleotide
gggagagaggaagagggatgggctctacgtcgatcgaatttcggatgcatcccaaataccagcataacccagaggtcgatagtactggatcccaagggcggcuuugccaagugc

SEQ ID NO: 22 - DNA, 48 bp, Artificial Sequence
  Description of Artificial Sequence Synthetic oligonucleotide
tggtggtggtggttgtggtggtggtggaagggcggcuuugccaagugc

SEQ ID NO: 23 - RNA, 23 nt, Artificial Sequence
  Description of Artificial Sequence Synthetic oligonucleotide
uucccgccgaaacgguucacgaa

SEQ ID NO: 24 - DNA, 153 bp, Artificial Sequence
  Description of Artificial Sequence Synthetic polynucleotide
tggtggtggtggttgtggtggtggtgggccuggccuccugcagugccacgcuaugcacuugggaaagccgcccuaguuggacacuccaugugguagaguaagggcggcuuugccaagugcuuagugcggcacaugcuuaccagcucucuaggc

SEQ ID NO: 25 - RNA, 22 nt, Artificial Sequence
  Description of Artificial Sequence Synthetic oligonucleotide
cacaugcuuaccagcucuaggc

SEQ ID NO: 26 - DNA, 46 bp, Artificial Sequence
  Description of Artificial Sequence Synthetic oligonucleotide
tggtggtggtggttgtggtggtggtggucaagaagccaaggauaau